(19) United States
(12) Patent Application Publication
(10) Pub. No.: US 2024/0358712 A1
(43) Pub. Date: Oct. 31, 2024

(54) COMPOSITIONS AND METHODS FOR IMPROVING SEXUAL SENSORY DISORDERS

(71) Applicant: WINSANTOR, INC., San Diego, CA (US)

(72) Inventor: Stanley Kim, San Diego, CA (US)

(21) Appl. No.: 18/767,228

(22) Filed: Jul. 9, 2024

Related U.S. Application Data
(63) Continuation of application No. PCT/US2023/010665, filed on Jan. 12, 2023.
(60) Provisional application No. 63/300,008, filed on Jan. 16, 2022.

(51) Int. Cl. A61K 31/5513; A61P 15/10

(52) U.S. Cl. CPC A61K 31/5513; A61P 15/10

(57) ABSTRACT

The present disclosure generally relates to a method and topical composition for treating sensory problems associated with sexual function of subjects, or more specifically, disruptions to the sensory function of the anogenitals of subjects. In one embodiment, the present disclosure relates to a topical, transdermal or transmucosal formulation for improving anogenital sensory disorders wherein the formulation comprises pirenzepine.

### INCORPORATION BY REFERENCE TO ANY PRIORITY APPLICATIONS

This application is a continuation of Int. App. No. PCT/US2023/010665 filed on Jan. 12, 2023, which was published in the English language and claims the benefit of priority to U.S. Provisional Patent Application No. 63/300,008, filed on Jan. 16, 2022. The disclosure of each of the above-referenced applications is incorporated by reference in its entirety.

### BACKGROUND

### Field

The present disclosure generally relates to a method and topical composition for treating sensory problems associated with sexual function of subjects, or more specifically, disruptions to the sensory function of the anogenitals of subjects. More specifically, the present disclosure relates to a topical, transdermal or transmucosal formulation for improving anogenital sensory disorders wherein the formulation comprises pirenzepine.

### Description of the Related Art

The penis and the vulva are highly sensitive organs in men and women, respectively, that contains thousands of nerve ending. In order for sexual arousal to occur, these nerve endings need to be stimulated properly. When a disruption of sensation occurs, it can be more difficult for men and women to become aroused, reach orgasm or ejaculation, and enjoy sexual activity. This disruption can be a change to an individual that is sudden/acute, for example from disease or injury, or gradual/chronic, as observed with aging. This disruption can lead to loss of sensitivity/hyposensitivity, hypersensitivity or pain, or other sensory problems or disorders, each that can lead to an inability to enter relationships, cause strain in relationships, reduce a partner's sexual satisfaction, and cause psychological damage to sufferers.

For hyposensitivity, many factors can lead to reduced penis or vulva sensitivity. Poor circulation attributable to poor cardiovascular health, or neuropathy, including peripheral neuropathy caused by systemic disorders such as diabetes (also known as diabetic neuropathy) or by hyperglycemia-induced glycation, the effect of certain medications, or traumatic injury. Health issues such as diabetes, lupus, multiple sclerosis, cancer, and its treatments such as prostate cancer, vascular diseases and its treatments such as statins, Peyronie's, and other biological/chemical causes. Other factors that can lead to reduced sensitivity include aging, lack of exercise, and obesity. Drug and alcohol use, smoking, use of prescription medication such as anti-depressants, low testosterone and aging are also thought to contribute to a loss of sensation. Additionally, damage to the organ can lead to impaired sensation, for example, penile sensation in men can be impaired from repeated masturbation or circumcision due to the removal of highly sensitive foreskin tissue.

In addition, of the main categories of male sexual dysfunction, delayed ejaculation (DE) has received the least attention even though it can lead to marked distress or interpersonal difficulties. DE is associated with sensory problems of the genitals, and is one of the diminished ejaculatory disorders, which range from varying delays in ejaculatory latency to a complete inability to ejaculate, and include DE, retrograde ejaculation (RE), anejaculation, anorgasmia and painful ejaculation.

Efforts have mostly focused on improving genital sensitivity by optimizing aspects of health, such as reducing body weight and quitting smoking. For circumcised men, foreskin restoration options may be available, however such procedures have mixed results and unwanted side effects. Surgical restoration procedures may be highly risky and be prohibitively expensive for most men. Topical creams are also available for increasing penis sensitivity due to the ease of application and low cost compared with other treatment options. Examples of sensitization creams are disclosed in U.S. Pat. Nos. 9,821,021 and 9,833,488.

Improving blood flow to the penis can also enhance its function, improve symptoms of erectile dysfunction and enhance sexual arousal. There are several drugs that improve blood flow including PDE5 inhibitors such as sildenafil and tadalafil. U.S. Pat. No. 5,439,938 discloses a method for treating male comprising a compound that generates nitric oxide in an amount sufficient to initiate penile erection. Also, infusion of L-arginine, a precursor to nitric oxide in the body, promotes vasodilation and blood flow (See Morikawa et al, Stroke 1994; 25:429-435). Topical delivery of arginine to produce enhanced blood flow in the penis is disclosed in U.S. Pat. No. 7,914,814. Increasing blood flow, however, is not the same as improving sensory problems.

Diabetic neuropathies are a family of nerve disorders caused by diabetes. People with diabetes can, over time, develop nerve damage throughout the body, and nerve problems can occur in every organ system, including the digestive tract, heart, and sex organs. About 60 to 70 percent of people with diabetes have some form of neuropathy, and the neuropathy can cause peripheral neuropathy in the feet and hands and can result in changes in sexual response, such an inability to have erections or reach sexual climax. Increasing sensitivity of the remaining nerves of neuropathy patients may result in increased sensation, ease the symptoms of neuropathy, and greatly improve a person's quality of life.

Hypersensitivity or hyperalgesia can lead to pain. Many of the same causes that cause hyposensitivity can also lead to hypersensitivity.

In women, vulvodynia is a complex gynecological disorder characterized by chronic pain localized to the vulva. It is a potentially debilitating condition that can last for years, cause physical disability, sexual dysfunction, and psychological difficulties. Daily activities and quality of life can be significantly impaired with many sufferers having trouble walking or sitting for long periods, sensitivity to clothing touching the vaginal area, and mild to intense pain typically described as burning, stinging, or itching. Although often difficult to diagnose, it is typically estimated that over 15 percent of the adult female population in western countries may experience vulvodynia at some point during their lifetime. It most commonly affects women of childbearing age.

The most common form of vulvodynia is vulvar vestibulitis. Women with vulvar vestibulitis typically experience pain involving and limited to the vestibule and only during or after touch or pressure is applied. Vulvar vestibulitis is characterized by pain, tenderness, vestibular erythema, itching, swelling and urethritis. The pain may be described as sharp, burning, or a sensation of rawness. Generalized vulvodynia is characterized by diffuse pain and/or a burning sensation on or around the vulva, the labia majora, labia minor, and/or the vestibule. The pain can be constant or intermittent and the symptoms, although not necessarily caused by touch or pressure to the vulva, can be exacerbated by physical contact to the area.

The etiology of vulvodynia is unknown. However, it has been hypothesized that viral, fungal, and bacterial assaults, allergic reactions, neuropathic processes, and an autoimmune response may play a role. Irritation of the muscles that support the uterus, bladder, and rectum (pelvic floor muscle or levator ani myalgia) as well as irritation of the nerves of the vulval tissue, known as pudendal neuralgia, may result in additional painful symptoms associated with vulvodynia.

Pudendal neuralgia (or also known as chronic perineal pain) is a term referring to chronic pain within the distribution of the pudendal nerve, which comprises, in women, the vulva as well as the labia majora and the skin around the anus as well as part of the mons veneris. The pudendal nerve also innervates the urethra and the anal mucosa. Thus, the dysfunction of the pudendal nerve extends not only to the sensory function of the perineal skin (including the vulva) but also to voiding and defecation. Symptoms such as the constant urge to void or the sensation of a foreign body in the rectum can occur. These symptoms are not classified as pain per se but nevertheless contribute to the individual's disability.

Pelvic pain is very common in men, with estimates of about 1 in 9 men suffering from pain across the underside somewhere between the pubic bone and tailbone. This is called the “perineal region”. The main nerve that provides sensation and muscle control in the perineal region is the pudendal nerve. In addition, in men, penile pain can affect the base, shaft, or head of the penis. It can also affect the foreskin. An itching, burning, or throbbing sensation may accompany the pain. Penile pain can be a result of an accident or disease. It can affect males of any age. The pain can vary depending on what underlying condition or disease is causing it. Some causes of pain in the penis include Peyronie's disease, priapism (resulting from, for example, side effects of drugs used to treat erection problems or drugs used to treat depression, blood clotting disorders, mental health disorders, blood disorders, such as leukemia or sickle cell anemia, alcohol or drug use, injury to the penis or spinal cord), Balantis, Phimosis and paraphimosis, sexually transmitted diseases, injuries to the penis, certain cancers, and certain skin diseases such as psoriasis.

Because of the potential for multiple causes, chronic pain can be difficult to treat. First-line therapy typically involves the treatment of suspected causes by pharmacologic treatment of infections and the discontinued use of suspected irritants and therapeutic agents that may contribute to the problem. Oral medications such as antihistamines, and tricyclic antidepressants, oral supplements such as calcium citrate, physical therapy, and dietary changes may provide some symptomatic relief. More invasive treatments include interferon intralesional injections, laser therapy and surgery, however these options are costly and may be associated with complications such as hematoma, wound dehiscence and uneven healing. There is no known cure as such for chronic pain syndromes.

### SUMMARY

Some embodiments provide a topical composition for improving the sensitivity of the sex organ of a subject comprising pirenzepine or a pharmaceutically acceptable salt in a topical carrier.

In some embodiments, the composition further comprises one or more pharmaceutically acceptable excipients.

One embodiment is a method for improving a sexual sensory disorder of a subject by applying to the anogenitals of a subject an effective amount of a composition comprising a therapeutically effective amount of pirenzepine, or a pharmaceutically acceptable salt thereof.

### DETAILED DESCRIPTION

While certain embodiments of the present disclosure have been shown and described herein, it will be obvious to those skilled in the art that such embodiments are provided by way of example only. Numerous variations, changes, and substitutions will now occur to those skilled in the art without departing from the disclosure. Various alternatives to the embodiments described herein may be employed. It is intended that methods and structures within the scope of the appended claims and their equivalents be covered thereby.

Unless defined otherwise, all technical and scientific terms used herein have the same meaning as is commonly understood by one of skill in the art to which this disclosure belongs. All patents and publications referred to herein are incorporated by reference.

As used in the specification and claims, the singular form “a”, “an” and “the” includes plural references unless the context clearly dictates otherwise.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range may vary from, for example, between 1% and 15% of the stated number or numerical range. The term “comprising” (and related terms such as “comprise” or “comprises” or “having” or “including”) includes those embodiments, for example, an embodiment of any composition of matter, composition, method, or process, or the like, that “consist of” or “consist essentially of” the described features.

Abbreviations used herein have their conventional meaning within the chemical and biological arts.

As used herein, the term “subject” or “patient” means any target of administration. The subject can be a vertebrate, for example, a mammal. Thus, the subject can be a human. The term does not denote a particular age or sex. Thus, adult, juvenile, and newborn subjects, whether male or female, are intended to be covered. A patient refers to a subject afflicted with a disease or disorder. The term “patient” includes human and veterinary subjects.

As used herein, the term “anogenital region” or “anogenital” refers to the region of the anus and the genitalia. In certain embodiments, the female anogenital region comprises the external genitalia, cervix, vagina, vulva and vulvar skin, labium, introitus, clitoris, urethral meatus, urethral fold, vulval vestibule, perineum, and/or anus. In certain embodiments, the male anogenital region comprises the penis, base of the penis, foreskin, urethral meatus, scrotum, perineum, and anus. As used herein, the term “urogenital region” refers to the region of the distal urinary tract and the genitalia. In some subjects, the anogenital and/or urogenital regions may be indistinct, intersex, or transitioning from male to female or female to male due to iatrogenic (for example, surgery or hormone therapy) or natural/genetic causes.

As used herein, the terms “treatment” and “treating,” or “improve”, “improvement”, and “improving” are used interchangeably herein. These terms refer to an approach for obtaining beneficial or desired results including but not limited to therapeutic benefit and/or a prophylactic benefit. By therapeutic benefit is achieved through the eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. It also refers to the restoration of lost function or sensation. For prophylactic benefit, the compositions may be administered to a patient at risk of developing a particular disease, or to a patient reporting one or more of the physiological symptoms of a disease, even though a diagnosis of this disease may not have been made. The compositions may be administered to a subject to prevent progression of physiological symptoms or to prevent progression of the underlying disorder. For example, in reference to the present disclosure, these terms refer to prevention and/or reversal of sensory disorders/problems associated with sexual function of subjects, or more specifically, disruptions to the sensory function of the anogenitals of subjects. This includes biological improvement such as reversing, preventing or slowing nerve degeneration; or improvement to functional symptoms such as reversing, preventing or slowing hyposensitivity, hypersensitivity, diminished ejaculatory disorders which range from varying delays in ejaculatory latency to a complete inability to ejaculate and include DE, retrograde ejaculation (RE), anejaculation, anorgasmia and painful ejaculation, and sexual function symptoms such as reduced ability to be aroused or remain aroused or intensity of arousal, reduced potency/intensity of or ability to reach orgasm or ejaculation, longer refractory ejaculation; or improvements to normal sexual activity including unperceived or latent sexual function changes that occur with aging. In one embodiment, the present disclosure contemplates the use of the disclosed formulation on other erogenous regions such as nipples.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof, for example, preventing and/or reversing peripheral neuropathy, or improving one or more sexual functions or dysfunctions.

The term “effective amount” or “therapeutically effective amount” refers to that amount of a compound described herein that is sufficient to affect the intended application including but not limited to disease treatment, as defined below. The therapeutically effective amount may vary depending upon the intended application (in vitro or in vivo), at a reasonable benefit/risk ratio applicable to any medical treatment, the severity of the disease condition, the manner of administration, or the subject and disease condition being treated, for example, the weight and age of the subject, and the like, which can readily be determined by one of ordinary skill in the art. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “pharmaceutically acceptable salt” refers to those salts which are suitable for pharmaceutical use, preferably for use in the tissues of humans and lower animals without undue irritation, allergic response and the like. Pharmaceutically acceptable salts of amines, carboxylic acids, and other types of compounds, are well known in the art. For example, S. M. Berge, et al., describe pharmaceutically acceptable salts in detail in J Pharmaceutical Sciences, 66: 1-19 (1977), incorporated herein by reference. The salts can be prepared in situ during the final isolation and purification of the compounds of the disclosure, or separately by reacting a free base or free acid function with a suitable reagent, as described generally below. For example, a free base function can be reacted with a suitable acid. Furthermore, where the compounds of the disclosure carry an acidic moiety, suitable pharmaceutically acceptable salts thereof may, include metal salts such as alkali metal salts, for example, sodium or potassium salts; and alkaline earth metal salts, for example, calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzoate, bisulfate, borate, butyrate, camphorate, camphorsulfonate, citrate, cyclopentanepropionate, digluconate, dodecylsulfate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, nicotinate, nitrate, oleate, oxalate, palmitate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed by direct reaction with the drug carboxylic acid or by using counterions such as halide, hydroxide, carboxylate, sulfate, phosphate, nitrate, sulfonate and aryl sulfonate:

A pharmacological agent may be formulated into pharmaceutical compositions by combination with appropriate, pharmaceutically acceptable carriers. By “pharmaceutically acceptable carrier” or “pharmaceutically acceptable excipient” is meant a component such as a carrier, diluent, excipient, solvent, enhancers, and the like of a composition that is compatible with the one or more pharmacological agents and other optional ingredients of the subject pharmacological agent compositions in that a pharmaceutically acceptable carrier may be combined with the pharmacological agent(s) without eliminating the biological or therapeutically effective activity of the one or more pharmacological agents (for example, pirenzepine), and is suitable for use in subjects as provided herein without undue adverse side effects (such as toxicity, irritation, allergic response, and death). Side effects are “undue” when their risk outweighs the benefit provided by the pharmaceutical agent. Supplementary or other active ingredients can also be incorporated into the compositions.

The term “co-administration,” “administered in combination with,” and their grammatical equivalents, as used herein, encompasses administration of two or more agents to an animal so that both agents and/or their metabolites are present in the animal at the same time. Co-administration includes simultaneous administration in separate compositions, administration at different times in separate compositions, or administration in a composition in which both agents are present.

“Localized treatment” as used herein refers to treatment of disruption of sensitivity of anogenitals of subjects wherein the drug is delivered locally and is not delivered via systemic delivery. This may include many different local areas or a few different local areas within, for example, treatment of skin, wherein the drug may be applied to many different locations or a few different locations on the skin, and wherein drug is delivered to tissues within and adjacent to the skin by absorption through the skin.

“Local delivery” as used herein refers to drug compound being carried to the site of therapeutic use. It includes, for example, applying a formulation directly to area of skin that is being treated, spraying a formulation to an area of skin being treated, spraying, or using a suppository or tampon to deliver a formulation intracavity to administer drug to the vaginal or anal passages, or a wipe or pad/patch to apply a formulation to the skin of the anogenital region (for example, penis, vulva or anus).

“Local tissue concentration” as used herein, refers to the concentration of pirenzepine within the tissue area to which treatment has been delivered and absorbed.

The present disclosure provides compositions and methods for treating disruption of anogenital sensation. In one embodiment, the present disclosure provides compositions and methods for improving the sensitivity of the anogenitals. In another embodiment, the present disclosure provides compositions and methods for treating hypersensitivity of the anogenitals of a subject, including treating chronic vulval and perineal pain, and chronic pelvic and penile pain; and symptoms and conditions associated with such pain. More particularly, provided herein are compositions comprising pirenzepine, wherein the composition is formulated for topical, transdermal or transmucosal administration.

Methods and compositions of the disclosure are applicable for the treatment of a variety of conditions and symptoms associated with disruption of normal anogenital sensation including, but not limited to, anogenital hyposensitivity or hypersensitivity, penile numbness, delayed ejaculation or orgasm, vulva numbness or reduced sensitivity, or alternatively, vulvodynia, pudendal neuralgia, pelvic floor tension myalgia, and the pain associated with any of these conditions or symptoms. The vulvodynia may be localized or generalized vulvodynia. The condition may be selected from, for example, vulval vestibulitis, localized provoked vestibulodynia (LPV), dysesthetic vulvodynia, vulvar dermatoses or cyclic vulvovaginitis. In one embodiment, the methods and compositions described herein are useful for the treatment of diminished ejaculatory disorders, and in particular, varying delays in ejaculatory latency to a complete inability to ejaculate due to sensory problems, and include delayed ejaculation (DE), anejaculation, anorgasmia and painful ejaculation.

Pirenzepine, an M1 selective antagonist, is used in the treatment of peptic ulcers, as it reduces gastric acid secretion and reduces muscle spasm. It is in a class of drugs known as muscarinic receptor antagonists—acetylcholine being the neurotransmitter of the parasympathetic nervous system which initiates the rest-and-digest state (as opposed to fight-or-flight), resulting in an increase in gastric motility and digestion; whereas pirenzepine would inhibit these actions and cause decreased gastric motility leading to delayed gastric emptying and constipation. It has no effects on the brain and spinal cord as it cannot diffuse through the blood-brain barrier. Pirenzepine has been investigated for use in myopia control. It is currently being investigated to treat peripheral neuropathy (see, WIPO Patent Application WO/2012/055018A1 Therapeutic Compositions for Diabetic Symmetrical Polyneuropathy, WIPO Patent Application WO/2015/089664A1 Methods and Compositions for Treatment Of Peripheral Neuropathies, and WO/2020/198252A1 Topical Formulations for Treatment of Peripheral Neuropathies, each incorporated herein by reference).

The term “pharmaceutically acceptable salts” refers to salts that are suitable for use in contact with human or animal tissues without undue toxicity, irritation, allergic response, etc. within the scope of sound medical judgment, and provides reasonable benefit/risk ratio. “Pharmaceutically acceptable salt” means any at least substantially non-toxic salt or ester salt of a compound disclosed herein that, upon administration to a recipient, is capable of providing, either directly or indirectly, a compound disclosed herein or a metabolite or residue thereof with inhibitory activity.

Pharmaceutically acceptable salts are well known in the art. For example, pharmaceutically acceptable salts are described by S. M. Berge et al. in detail in J. Pharmaceutical Sciences, 1977, 66, 1-19, which is incorporated herein by reference. Pharmaceutically acceptable salts of the compounds disclosed herein include those derived from suitable inorganic and organic acids, including pirenzepine free base or pirenzepine monohydrate. Examples of pharmaceutically acceptable non-toxic acid addition salts are amino salts which are formed with mineral acids such as hydrochloric acid (for example, pirenzepine dihydrochloride or monohydrochloride), hydrobromic acid, phosphoric acid, sulfuric acid and perchloric acid, or with organic acids such as acetic acid, oxalic acid, maleic acid, tartaric acid, citric acid, succinic acid or malonic acid, or formed by other methods in the art such as ion exchange. Other pharmaceutically acceptable salts include adipates, alginates, ascorbates, aspartates, benzenesulfonates, benzoates, bisulfates, borates, butyrates, camphorate, camphorsulfonates, citrates, cyclopentane propionates, digluconates, dodecyl sulfates, esylates, formates, fumarates, glucoheptonates, glycerophosphates, gluconate, hemisulfate, heptanoate, hexanoate, hydroiodide, 2-hydroxy-ethanesulfonate, lactobionate, lactate, laurate, lauryl sulfate, malates, maleates, malonates, methanesulfonates, 2-naphthalenesulfonates, nicotinates, nitrates, oleates, oxalates, palmitates, pamoate, a salt of pectinic acid, persulfates, 3-phenylpropionates, phosphates, picrates, pivalates, propionates, stearates, succinates, sulfates, tartrates, thiocyanates, p-toluenesulfonates, undecanoates, valerates, and the like.

The formulations described herein are suitable for localized treatment of sensory disorders of the anogenital region of a subject. The formulations contain pirenzepine in a composition suitable for topical delivery to a subject. Compositions may include gel, cream, lotion, solution, suspension, emulsion, ointment, powder, crystalline forms, spray, foam, salve, paste, plaster, paint, bioadhesive, suppository, tampon, body wipes, wound dressings, skin patches, genital pads, transdermal patches, and the like. Formulations may further include additional ingredients such as ingredients to facilitate delivery of the active compounds, enhance the therapeutic effect, have a secondary effect, or minimize side effects. Such formulations allow for efficacious delivery of pirenzepine to the site of administration, such as but not limited to penis, vagina and the surrounding tissue and organs.

The topical compositions may be formulated as, for example, a gel, lotion, cream, mousse, aerosol, ointment, or lubricants, etc., so long as when the composition is applied, especially to the genitalia, the formulation will substantially stay in place. For example, there will not be substantial run-off, for a sufficient time after application, to permit an individual to spread the composition over a relevant portion of the penis, including over the glans of the penis, or over a relevant portion of the vagina, including over the vulva of the vagina. It can also be formulated to dry relatively quickly and penetrate the skin of the subject. Other factors to consider include transference (or the non-transference) to the sexual partner, the ability to wash off any residual formulation, and the chronic effect of the formulation over repeated use.

In some embodiments, the topical composition provided herein may comprise a pharmaceutical carrier, diluent, co-solvent, emulsifier, penetration enhancer, preservative, emollient, or a combination thereof. Acceptable carriers or diluents for therapeutic use are well-known in the pharmaceutical art, and are described, for example, in Florence A T and Siepmann J, editors. Modern Pharmaceutics Volume 1: Basic Principles and Systems. 5^{th} ed. CRC Press, 2009; Augsburger L L and Hoag S W, editors. Pharmaceutical Dosage Forms: Tablets. 3^{rd} ed. CRC Press, 2016; Ansel H C. Introduction to Pharmaceutical Dosage Forms, 2^{nd} ed. Lea & Febiger, 1976; Adejare A, editor. Remington: The Science and Practice of Pharmacy, 23^{rd} ed. Academic Press, 2020; Brunton L, et al., editors. Goodman & Gilman's The Pharmacological Basis of Therapeutics, 13^{th} ed. McGraw-Hill, 2017; Shah V P et al., editors. Topical Drug Bioavailability, Bioequivalence, and Penetration, 2^{nd} ed. Springer, 2015; Hillery A M, & Park K. Drug Delivery and Targeting: For Pharmacists and Pharmaceutical Scientists, CRC Press, 2016; Allen L. Pharmaceutical Dosage Forms and Drug Delivery Systems, 11^{th} ed. LWW, 2017 (each incorporated herein by reference).

### Excipients

The pharmaceutical compositions may include one or more inert excipients, which include water, buffered aqueous solutions, surfactants, volatile liquids, starches, polyols, granulating agents, microcrystalline cellulose, diluents, lubricants, acids, bases, salts, emulsions, such as oil/water emulsions, oils such as mineral oil and vegetable oil, wetting agents, chelating agents, antioxidants, sterile solutions, complexing agents, disintegrating agents and the like. The CTFA Cosmetic Ingredient Handbook, Eighth Edition, 2000 and the Tenth Edition, 2004, which is incorporated by reference herein in its entirety, describes a wide variety of cosmetic and pharmaceutical ingredients commonly used in skin care compositions, which are suitable for use in the compositions of the present disclosure. Examples of these functional classes disclosed in this reference include: absorbents, abrasives, anticaking agents, antifoaming agents, antimicrobial agents, antioxidants, binders, biological additives, buffering agents, bulking agents, chelating agents, chemical additives, colorants, cosmetic astringents, cosmetic biocides, denaturants, drug astringents, external analgesics, film formers, fragrance components, humectants, opacifying agents, pH adjusters, plasticizers, preservatives, reducing agents, skin bleaching agents, skin-conditioning agents (emollient, humectants, miscellaneous, and occlusive), skin protectants, solvents, foam boosters, hydrotropes, solubilizing agents, steroidal anti-inflammatory agents, surfactants/emulsifying agents, suspending agents (nonsurfactant), sunscreen agents, topical analgesics, ultraviolet light absorbers, SPF boosters, thickening agents, waterproofing agents, and viscosity increasing agents (aqueous and nonaqueous).

Surfactants which can be used to form pharmaceutical compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

One surfactant may be the sodium salt form of the compound, which may include the monosodium salt form. Suitable sodium salt surfactants may be selected based on desirable properties, including high speed of polymerization, small resultant particle sizes suitable for delivery, good polymerization yields, stability including freeze-thaw and shelf-life stability, improved surface tension properties, and lubrication properties.

The surfactant may be any suitable, non-toxic compound that is non-reactive with the medicament and that substantially reduces the surface tension between the medicament, the excipient and the site of administration. The surfactants include but are not limited to: oleic acid available under the tradenames Mednique 6322 and Emersol 6321 (from Cognis Corp., Cincinnati, Ohio); cetylpyridinium chloride (from Arrow Chemical, Inc. Westwood, N.J.); soya lecithin available under the tradename Epikuron 200 (from Lucas Meyer Decatur, Ill.); polyoxyethylene(20) sorbitan monolaurate available under the tradename Tween 20 (from ICI Specialty Chemicals, Wilmington, Del.); polyoxyethylene(20) sorbitan monostearate available under the tradename Tween 60 (from ICI); polyoxyethylene(20) sorbitan monooleate available under the tradename Tween 80 (from ICI); polyoxyethylene (10) stearyl ether available under the tradename Brij 76 (from ICI); polyoxyethylene (2) oleyl ether available under the tradename Brij 92 (from ICI); Polyoxyethylene-polyoxypropylene-ethylenediamine block copolymer available under the tradename Tetronic 150 R1 (from BASF); polyoxypropylene-polyoxyethylene block copolymers available under the tradenames Pluronic L-92, Pluronic L-121 end Pluronic F 68 (from BASF); castor oil ethoxylate available under the tradename Alkasurf CO-40 (from Rhone-Poulenc Mississauga Ontario, Canada); and mixtures thereof.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical, and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

Surfactants may be used in any formulation described herein where its use is not otherwise contradicted. In some embodiments, the use of no surfactants or limited classes of surfactants is desirable. The topical formulations described herein can contain no, or substantially no surfactant, i.e., contain less than approximately 0.0001% by weight of surface-active agents. This is particularly the case if one employs a cromone as described above. If desired, however, the formulations can contain surface-active agents conventionally employed in topical formulations, such as oleic acid, lecithin, sorbitan trioleate, cetylpyridinium chloride, benzalkonium chloride, polyoxyethylene (20) sorbitan monolaurate, polyoxyethylene (20) sorbitan monostearate, polyoxyethylene (20) sorbitan monooleate, polyoxypropylene/polyoxyethylene block copolymers, polyoxypropylene/polyoxyethylene/ethylenediamine block copolymers, ethoxylated castor oil and the like, where the proportion of surface-active agents, if present, can be about 0.0001 to 1% by weight, in particular about 0.001 to 0.1% by weight, based on the total formulation. Other suitable surfactant/emulsifying agents would be known to one of skill in the art and are listed in the CTFA International Cosmetic Ingredient Dictionary and Handbook, Vol. 2, 7^{th} Edition (1997).

Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propylp-hydroxybenzoate.

Chelating agents which can be used to form pharmaceutical compositions and dosage forms include, but are not limited to, ethylene diaminetetraacetic acid (EDTA), EDTA disodium, calcium disodium edetate, EDTA trisodium, albumin, transferrin, desferoxamine, desferal, desferoxamine mesylate, EDTA tetrasodium and EDTA dipotassium, sodium metasilicate or combinations of any of these. In some embodiments, up to about 0.1% W/V of a chelating agent, such as EDTA or its salts, is added to the formulations described herein.

Preservatives which can be used to form pharmaceutical compositions and dosage forms include, but are not limited to, purite, peroxides, perborates, imidazolidinyl urea, diazolidinyl urea, phenoxyethanol, alkonium chlorides including benzalkonium chlorides, methylparaben, ethylparaben and propylparaben. In other embodiments, suitable preservatives for the compositions include: benzalkonium chloride, purite, peroxides, perborates, thimerosal, chlorobutanol, methyl paraben, propyl paraben, phenylethyl alcohol, edetate disodium, sorbic acid, Onamer M, or other agents known to those skilled in the art. In some embodiments, such preservatives may be employed at a level of from 0.004% to 0.02% W/V. In some compositions of the present application the preservative, for example, benzalkonium chloride, methyl paraben, and/or propyl paraben, may be employed at a level of from about 0.001% to less than about 0.01%, for example, from about 0.001% to about 0.008%, or about 0.005% W/V. It has been found that a concentration of benzalkonium chloride of about 0.005% may be sufficient to preserve the compositions of the present disclosure from microbial attack. One of skill in the art could determine the proper concentration of ingredients as well as combinations of various ingredients for generating a suitable topical formulation. For example, ophthalmic drops or formulations for application to skin may use a mixture of methyl and propyl parabens at about 0.02% W/V and about 0.04% W/V respectively. In some embodiments, these formulations use methyl paraben and/or propyl paraben in amounts up to about 0.02% W/V and up to about 0.04% W/V respectively, which encompasses the embodiments where no methyl paraben or no propyl paraben is used.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (for example, peanut oil, cottonseed oil, sunflower oil, sesame oil, olive oil, corn oil, and soybean oil), zinc stearate, ethyl oleate, ethyl laureate, agar, or mixtures thereof.

Thickening agents which can be used to form pharmaceutical compositions and dosage forms include, but are not limited to, isopropyl myristate, isopropyl palmitate, isodecyl neopentanoate, squalene, mineral oil, C12-C15 benzoate and hydrogenated polyisobutene. Those agents which would not disrupt other compounds of the final product, such as non-ionic thickening agents may be desirable. The selection of additional thickening agents is well within the skill of one in the art.

Skin conditioning agents can be emollients, humectants, and moisturizers. A humectant is a moistening agent that promotes retention of water due to its hygroscopic properties. Suitable skin conditioning agents include urea; guanidine; aloe vera; glycolic acid and glycolate salts such as ammonium and quaternary alkyl ammonium; lactic acid and lactate salts such as sodium lactate, ammonium lactate and quaternary alkyl ammonium lactate; polyhydroxy alcohols such as sorbitol, glycerol, mannitol, xylitol, hexanetriol, propylene glycol, butylene glycol, hexylene glycol, polymeric glycols such as polyethylene glycol and polypropylene glycol; carbohydrates such as alkoxylated glucose; starches; starch derivatives; glycerin; pyrrolidone carboxylic acid (PCA); lactamide monoethanolamine; acetamide monoethanolamine; volatile silicone oils; nonvolatile silicone oils; and mixtures thereof. Suitable silicone oils can be polydialkylsiloxanes, polydiarylsiloxanes, polyalkarylsiloxanes and cyclomethicones having 3 to 9 silicon atoms.

An emollient is an oleaginous or oily substance which helps to smooth and soften the skin, and may also reduce its roughness, cracking or irritation. Typical suitable emollients include mineral oil having a viscosity in the range of 50 to 500 centipoise (cps), lanolin oil, coconut oil, cocoa butter, olive oil, almond oil, macadamia nut oil, aloe extracts such as aloe vera lipoquinone, synthetic jojoba oils, natural sonora jojoba oils, safflower oil, corn oil, liquid lanolin, cottonseed oil and peanut oil. In some embodiments, the emollient is a cocoglyceride, which is a mixture of mono, di and triglycerides of cocoa oil, sold under the trade name of Myritol 331 from Henkel KGaA, or Dicaprylyl Ether available under the trade name Cetiol OE from Henkel KGaA or a C12-C15 Alkyl Benzoate sold under the trade name Finsolv TN from Finetex. Another suitable emollient is DC 200 Fluid 350, a silicone fluid, available from Dow Corning Corp, and caprylic acid, one of the fatty acids found in coconut oil, a medium-chain fatty acid believed to have potent antibacterial, antifungal, and anti-inflammatory properties.

Other suitable emollients include squalane, castor oil, polybutene, sweet almond oil, avocado oil, calophyllum oil, ricin oil, vitamin E acetate, olive oil, silicone oils such as dimethylopolysiloxane and cyclomethicone, linolenic alcohol, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C12-C15) alcohols, the octanoates and decanoates of alcohols and polyalcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients which are solids or semi-solids at ambient temperatures may be used. Such solid or semi-solid cosmetic emollients include glyceryl dilaurate, hydrogenated lanolin, hydroxylated lanolin, acetylated lanolin, petrolatum, isopropyl lanolate, butyl myristate, cetyl myristate, myristyl myristate, myristyl lactate, cetyl alcohol, isostearyl alcohol and isocetyl lanolate. One or more emollients can optionally be included in the formulation.

Anti-oxidants which can be used to form pharmaceutical compositions and dosage forms include, but are not limited to, propyl, octyl and dodecyl esters of gallic acid, butylated hydroxyanisole (BHA, usually purchased as a mixture of ortho and meta isomers), green tea extract, uric acid, cysteine, pyruvate, nordihydroguaiaretic acid, ascorbic acid, salts of ascorbic acid such as ascorbyl palmitate and sodium ascorbate, ascorbyl glucosamine, vitamin E (i.e., tocopherols such as a-tocopherol), derivatives of vitamin E (for example, tocopheryl acetate), retinoids such as retinoic acid, retinol, trans-retinol, cis-retinol, mixtures of trans-retinol and cis-retinol, 3-dehydroretinol and derivatives of vitamin A (for example, retinyl acetate, retinal and retinyl palmitate, also known as tetinyl palmitate), sodium citrate, sodium sulfite, lycopene, anthocyanids, bioflavinoids (for example, hesperitin, naringen, rutin and quercetin), superoxide dismutase, glutathione peroxidase, butylated hydroxytoluene (BHT), indole-3-carbinol, pycnogenol, melatonin, sulforaphane, pregnenolone, lipoic acid and 4-hydroxy-5-methyl-3[2H]-furanone.

Skin protecting agents are agents that protect the skin against chemical irritants and/or physical irritants, for example, UV light, including sunscreens, anti-acne additives, anti-wrinkle and anti-skin atrophy agents. Suitable sunscreens as skin protecting agents include 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomethyl salicylate, octyl salicylate, 4,4′-methoxy-t-butyldibenzoylmethane, 4-isopropy dibenzoylmethane, 3-benzylidene camphor, 3-(4-methylbenzylidene) camphor, anthanilates, ultrafine titanium dioxide, zinc oxide, iron oxide, silica, 4-N,N-(2-ethylhexyl)methylaminobenzoic acid ester of 2,4-dihydroxybenzophenone, 4-N,N-(2-ethylhexyl)-methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone and 4-N,N(2-ethylhexyl)-methylaminobenzoic acid ester of 4-(2-hydroxyethoxy)dibenzoylmethane. Suitable anti-acne agents include salicylic acid; 5-octanoyl salicylic acid; resorcinol; retinoids such as retinoic acid and its derivatives; sulfur-containing D and L amino acids other than cysteine; lipoic acid; antibiotics and antimicrobials such as benzoyl peroxide, octopirox, tetracycline, 2,4,4′-trichloro-2′-hydroxydiphenyl ether, 3,4,4′-trichlorobanilide, azelaic acid, phenoxyethanol, phenoxypropanol, phenoxisopropanol, ethyl acetate, clindamycin and melclocycline; flavonoids; and bile salts such as scymnol sulfate, deoxycholate and cholate. Examples of anti-wrinkle and anti-skin atrophy agents are retinoic acid and its derivatives, retinol, retinyl esters, salicylic acid and its derivatives, sulfur-containing D and L amino acids except cysteine, alpha-hydroxy acids (for example, glycolic acid and lactic acid), phytic acid, lipoic acid, and lysophosphatidic acid.

The formulations may also contain irritation-mitigating additives to minimize or eliminate the possibility of skin irritation or skin damage resulting from the permeation-enhancing base, drying agents or other components of the composition. Suitable irritation-mitigating additives include, for example: alpha-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the composition.

A dry-feel modifier is an agent which when added to an emulsion, imparts a “dry feel” to the skin when the emulsion dries. Dry feel modifiers can include talc, kaolin, chalk, zinc oxide, silicone fluids, inorganic salts such as barium sulfate, surface treated silica, precipitated silica, fumed silica such as an Aerosil available from Degussa Inc. of New York, N.Y. U.S.A. Another dry feel modifier is an epichlorohydrin cross-linked glyceryl starch of the type that is disclosed in U.S. Pat. No. 6,488,916.

In certain embodiments, compositions of the instant disclosure are formulated to have viscosity best suited for the target tissue (for example, anogenital region) and to mimic the properties of normal genital fluids. For example, compositions formulated as gels applied to mucous membranes may be designed to have viscosity values consistent with or similar to normal mucus, and exhibiting non-Newtonian, shear-thinning (pseudoplastic) flow properties. Standardized methodology for quantitative comparisons of over-the-counter vaginal products based features such as, stickiness, ropiness, peaking, rubberiness, thickness, smoothness, and slipperiness, are known in the art (Mahan et al., Contraception, 84:184, 2011). In some embodiments, compositions formulated as gels applied to mucous membranes may strengthen mucus quality and/or mucin coverage of the body surface. In certain implementations, the compositions may stimulate the production or proffer an acidic barrier in the urogentical and/or anogenital region with improved muco-adhesion, which may increase the bioavailablity of one or more active components of the composition and result in a beneficial impact on the genital microbiome as disclosed in N Peppas, et al., J Biomater Sci Polym Ed. 20 (2209): 1-20, hereby incorporated by reference in its entirety and particularly in relation to muco-adhesive carrier development.

For compositions applied to skin (such as the vulva, perineum, or penis) or inside the vagina, a viscosity-increasing agent can be added in an amount that allows the composition to spread easily to form a thin layer when minimal physical pressure is applied, and to have adequate viscosity and shear-thinning properties so that the composition does not “run” off or out of the genital tissue upon topical application. Mucoadhesive formulations that are retained at the genital surface (for example, vulvar, vaginal, penile, foreskin surface) for prolonged biological activity are known in the art (reviewed by Khutoryanskiy, Macromol. Biosci. 11:748, 2011; Brooks, Front. Chem. 3:65, 2015). Muco-adhesive formulas must have polymer compositions that actively admix and interact with physiologic mucin and mucus of secretions. Some common gelling agents do not intertwine with natural mucins and are therefore not muco-adhesive and are rapidly lost from the epithelium.

Compositions of the present disclosure may comprise a viscosity-increasing agent in an amount ranging from, for example, about 0.05% to about 10% by weight of the composition. In certain embodiments, the viscosity enhancing agent comprises a tensioactive cellulose or gum. Tensioactive celluloses and gums can also act to emulsify and pull particles and essential oils into solution. In certain embodiments, additional surfactants, which may have a harsh effect on cells, are not needed or included in the topical compositions. In certain embodiments, the viscosity-increasing agent comprises guar gum, methylcellulose, ethylcellulose, ethyl methyl cellulose, ethyl hydroxyethyl cellulose, carboxymethylcellulose, hydroxypropylcellulose, hydroxyethyl methyl cellulose, hydroxypropylmethylcellulose (hypromellose), hydroxyethylcellulose, cetyl hydroxyethycellulose, hydroxypropyl guar gum glycosaminoglycans (for example, hyaluronic acid), nonionic triblock copolymers such as poloxamers, gelatins, alginates, carrageenan, and agar, or any combination thereof. In some embodiments, the compositions may comprise a viscosity-increasing agent comprising glycosaminoglycans (for example, hyaluronic acid), nonionic triblock copolymers such as poloxamers, gelatins, carrageenan, agar, and combinations thereof.

In certain embodiments, the topical composition may further comprise a pH modifying agent to adjust the final pH of the composition to the target or desired pH. The pH modifying agent may comprise an acidifying agent, an alkalinizing agent, and/or both an acidifying agent and an alkalinizing agent. In certain embodiments, the pH modifying agent is in an amount ranging from about 0.01% to about 1%.

In certain embodiments, topical compositions of the present disclosure further comprise a buffering agent. The buffering agent may be in an amount ranging from about 0.01% to about 0.9% by weight of the composition. A buffering agent refers to a compound or a mix of compounds that, when present in a solution, resists changes in the pH of the solution when small quantities of acid or base are added or upon dilution with a solvent or bodily fluid. Buffer capacity is a measure of the resistance to change in the pH of a solution when acids or bases are added to the solution. The total amount of the buffering agent (for example, conjugate acid-base pair) is selected such that the pH of the composition is maintained at the desired pH or range of pH values. Thus, the greater the amount of the buffering capacity, the more resistant the pH of the composition is to change. In certain embodiments, a buffering agent contains an acidic species to neutralize hydroxide (OH⁻) ions and a basic species to neutralize hydrogen (H⁺) ions. However, the acidic and basic species of the buffering agent should not consume each other through a neutralization reaction.

In certain embodiments, the buffering agent is a simple buffered solution comprising a weak acid and a salt of the weak acid or a weak base and a salt of the weak base. Thus, the buffering agent can include a weak acid-base conjugate pair or weak base-acid conjugate pair. Examples of weak acid/salt of weak acid and weak base/salt of weak base parings include citric acid/sodium citrate, lactic acid/sodium lactate, acetic acid/sodium acetate, monosodium phosphate/disodium phosphate, propionic acid/sodium propionate, butyric acid/sodium butyrate, carbonic acid/sodium bicarbonate, malic acid/sodium malate, ascorbic acid/sodium ascorbate benzoic acid/sodium benzoate, succinic acid/sodium succinate and sodium borate/boric acid. In certain embodiments, the buffering agent comprises unrelated weak acid-base pairs. Examples of such combinations include disodium phosphate/citric acid, disodium phosphate/lactic acid, monosodium phosphate/sodium lactate, monosodium phosphate/sodium citrate, sodium citrate/lactic acid, sodium lactate/citric acid, monopotassium phosphate/citric acid, monopotassium phosphate/lactic acid, monopotassium phosphate/sodium lactate, monopotassium phosphate/sodium citrate, monopotassium citrate/lactic acid, and potassium lactate/citric acid. In addition, for multivalent anions, the calcium salt rather than sodium salt may be used (for example, calcium citrate). Example buffer can also include gluconolactone/gluconic acid.

In certain embodiments, the buffering agent is selected such that the buffering agent's acid form has a pKa the same as or close to the desired pH of the composition or a pH within the desired range of pH values, functions with a similar buffering capacity to surfaces and fluids that physiologically occur in anogenital region (for example, vaginal mucin-acidic barrier, cervico-vaginal secretions, semen, menses-flow, or a combination thereof), or maintains pH to that of the target epithelial surface (Rastogi et al., Contraception. 93:337, 2016). In certain embodiments, a buffering agent comprises a monocarboxylate, a dicarboxylate, a carboxylic acid, or a combination thereof. In some embodiments, a buffering agent may comprise an acetate, borate, citrate, fumarate, lactate, malate, malonate, nitrate, phosphate, propanoate, succinate, tartrate, tromethamine, or any combination thereof. In some embodiments, a buffering agent comprises lactic acid, sodium lactate, sodium phosphate (monobasic, dibasic, or both), potassium phosphate (monobasic, dibasic, or both), sodium citrate, potassium citrate, calcium citrate, acetic acid, sodium acetate, citric acid, disodium citrate, trisodium citrate, boric acid/sodium, succinic acid, sodium succinate, gluconolactone, disodium succinate, tartaric acid, sodium tartarate, sodium ascorbate, ascorbic acid, tromethamine (Tris), or any combination thereof. In certain embodiments, a buffering agent comprises citric acid and disodium phosphate, lactic acid and sodium lactate, gluconolactone, or mono- or disodium phosphate and lactic acid.

In one embodiment, topical compositions of the present disclosure are specifically isotonic to the target genital fluids or tissues that they will contact. Tonicity is a measure of the effective osmotic pressure gradient (as defined by the water potential of two solutions) of two solutions separated by a semipermeable membrane. Tonicity is commonly used when describing the response of cells immersed in an external solution. In other words, tonicity is the relative concentration of solutions that determine the direction and extent of diffusion from a fluid across cell membranes in tissue. Blood normally has an osmotic pressure that corresponds to that of a 0.9% solution of sodium chloride (approximately 280 mOsm/kg). However, osmotic pressure for physiologic fluids occurring in the anogenital region can vary widely, from the low end for cervico-vaginal fluids (for example, approximately 128 mOsm/kg), to approximately 280 mOsm/kg for menses blood, to a higher level for semen (for example, approximately 320+ mOsm/kg). A composition (for example, solution or gel) is considered isotonic when its tonicity matches that of the physiologic fluids it will contact. A composition is isotonic with a body fluid when the magnitude of the salts (ions) is equal between the composition and the physiologic fluid. Tonicity equilibrium is reached in physiologic fluids by water moving across cell membranes, but the salts and ions staying in their fluid of origin. A solution is isotonic with a living cell if there is no net gain or loss of water by the cell, or other changes in the cell ultrastructure, when it is in contact with said solution, even though individual water molecules may move freely across the cell membranes.

Hypertonic solutions cause a net movement of water out of the cells (as the water moves to create equilibrium with the high salt levels outside of the cell). This dehydration of the cell is concentration dependent and leads to osmotic stress which can increase reactive oxygen species, cause cytoskeletal rearrangement, and damage DNA and mitochondrial function within minutes of exposure. Most current genital products are hypertonic, resulting in epithelial cell and sperm death on contact. Hypotonic solutions cause a net flow of water into the cell and cause cell bursting and death. Some deviations of salt levels in physiologic fluids from the level found in blood and tissues may serve a purpose. For example, the lower osmolality of cervico-vaginal fluids that facilitates vaginal epithelial cell lysis and death as a part of normal vaginal function. In another example, the higher osmolality of semen can protect sperm cells from the lower osmolality of cervico-vaginal secretions following ejaculation in the vagina and admixing of fluids during vaginal intercourse.

Related to tonicity is osmosis, which is the movement of solvent across a semipermeable membrane from an area of higher solute concentration to an area of lower solute concentration to produce equilibrium. Osmotic pressure of a solution is the pressure that must be applied to stop the flow of solvent across a semipermeable membrane.

In certain embodiments, the compositions of the present disclosure further comprise an osmolality adjusting agent to adjust the tonicity of the compositions. Exemplary osmolality adjusting agents include electrolytes, mono- or disaccharides, inorganic salts (for example, sodium chloride, calcium chloride, potassium chloride, sodium sulfate, magnesium chloride), or a combination thereof. In some embodiments, an osmolality adjuster is glucose, sucrose, sodium chloride, potassium chloride, calcium chloride, sodium sulfate, magnesium chloride, dextrose, mannitol, or any combination thereof.

In certain embodiments, the osmolality range of the compositions disclosed herein ranges from about 120 mOsm/kg to about 450 mOsm/kg or from about 240 mOsm/kg to about 450 mOsm/kg. In certain embodiments, the osmolality of the compositions of the present disclosure is about 120 mOsm/kg, about 125 mOsm/kg, about 130 mOsm/kg, about 135 mOsm/kg, about 140 mOsm/kg, about 145 mOsm/kg, about 150 mOsm/kg, about 155 mOsm/kg, about 160 mOsm/kg, about 165 mOsm/kg, about 170 mOsm/kg, about 175 mOsm/kg, about 180 mOsm/kg, about 185 mOsm/kg, about 190 mOsm/kg, about 195 mOsm/kg, about 200 mOsm/kg, about 205 mOsm/kg, about 210 mOsm/kg, about 215 mOsm/kg, about 220 mOsm/kg, about 225 mOsm/kg, about 230 mOsm/kg, about 235 mOsm/kg, about 240 mOsm/kg, about 245 mOsm/kg, about 250 mOsm/kg, about 255 mOsm/kg, about 260 mOsm/kg, about 265 mOsm/kg, about 270 mOsm/kg, about 280 mOsm/kg, about 285 mOsm/kg, about 290 mOsm/kg, about 295 mOsm/kg, about 300 mOsm/kg, about 305 mOsm/kg, about 310 mOsm/kg, about 315 mOsm/kg, about 320 mOsm/kg, about 325 mOsm/kg, about 330 mOsm/kg, about 335 mOsm/kg, about 340 mOsm/kg, about 345 mOsm/kg, about 350 mOsm/kg, about 355 mOsm/kg, about 360 mOsm/kg, about 365 mOsm/kg, about 370 mOsm/kg, about 375 mOsm/kg, about 380 mOsm/kg, about 385 mOsm/kg, about 390 mOsm/kg, about 395 mOsm/kg, about 400 mOsm/kg, or about 450 mOsm/kg.

In certain embodiments, the topical composition is matched for tonicity (for example, salt/ion levels) to the normal, physiological genital fluid pH (for example, CVF, urethral secretions, semen, smegma) of the subject; anogenital tissue of the subject (for example, vaginal mucosa, anogenital skin); or at an appropriate tonicity for the particular method of use. In certain embodiments, the tonicity ranges from about 125 mOsm/kg to about 240 mOsmo/kg. Such embodiments match the hypotonic CVF which supports lysis of vaginal epithelial cells and vaginal “self-cleaning.” This cell lysis releases glycogen, which healthy genital microbiota utilize for growth and development. Such embodiments are ideal for delivery inside the vaginal canal. In other embodiments the tonicity ranges from about 240 mOsm/kg to about 280 mOsm/kg. Such embodiments match the tonicity of anogenital tissues and are ideal for contact with skin anogenital tissue surfaces. In a particular embodiment, the tonicity ranges from about 280 mOsmo/kg to about 450 mOsmo/kg to match the tonicity of semen as deposited in the vagina. In certain embodiments, tonicity may be expressed as mOsm/kg or mOsm/L. Osmolality is a measure of the osmoles (Osm) of solute per kilogram of solvent. Depending on the density of the solvent, the osmolality and osmolarity (Osm of solute per L of solution) may differ. In certain embodiments, the osmolality and osmolarity values are substantially interchangeable.

In certain embodiments, the topical, isotonic composition further comprises a solvent (for example, aqueous solvent, water) in an amount greater than about 88% (for example, ranging from about 88% to about 98%). In further embodiments, the solvent comprises water.

### Topical Penetration Enhancers

The delivery of drugs topically to the skin provides many advantages. For the patient, it is comfortable, convenient, and noninvasive. The variable rates of absorption and metabolism possibly encountered in oral treatment may be avoided, and other inherent inconveniences (for example, gastrointestinal irritation, the need for administration with food in some cases or without food in other cases) are eliminated. Such localized treatment avoids incurring high systemic drug levels and possible adverse effects that could follow.

The topical delivery of drugs into the skin, however, is commonly challenging. Skin is a structurally complex, relatively thick membrane. Molecules moving from the environment into and through intact skin must first penetrate the stratum corneum and any material on its surface. The stratum corneum is a layer approximately 10-15 micrometers thick over most of the body that consists of dense, highly keratinized cells. The high degree of keratinization within these cells, as well as their dense packing, are believed to be the factors most responsible for creating, in most cases, a substantially impermeable barrier to drug penetration. With many drugs, the rate of penetration through the skin is extremely low without the use of some means to enhance the skin's permeability. As the stratum corneum of many inflammatory dermatoses is commonly thicker than that of normal skin, the penetration of topical drugs into the affected areas of skin is particularly difficult to achieve.

To increase the degree and rate at which a drug penetrates the skin, various approaches have been followed, each of which involves the use of either a chemical penetration enhancer or a physical penetration enhancer. Physical enhancements of skin permeation include, for example, electrophoretic techniques such as iontophoresis. The use of ultrasound (or “phonophoresis”) as a physical penetration enhancer has also been researched. Chemical penetration enhancers are more commonly used. These are compounds that are topically administered along with a drug (or, in some cases, prior to drug administration) in order to increase the permeability of the stratum corneum, and thereby provide for enhanced penetration of the drug through the skin. Ideally, such chemical penetration enhancers (or “permeation enhancers,” as the compounds are referred to herein) are compounds that are innocuous and serve merely to facilitate diffusion of the drug through the stratum corneum.

Various compounds for enhancing the permeability of skin are known in the art and are described in the pertinent texts and literature. Compounds that have been used to enhance skin permeability include: sulfoxides such as dimethylsulfoxide (DMSO) and decylmethylsulfoxide (C10MSO); ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); the 1-substituted azacycloheptan-2-ones, particularly 1-n-dodecylcyclazacycloheptan-2-one (available under the trademark Azone® from Nelson Research & Development Co., Irvine, Calif.; see U.S. Pat. Nos. 3,989,816, 4,316,893, 4,405,616, and 4,557,934); alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as propylene glycol, ethylene glycol, glycerol, butanediol, polyethylene glycol, and polyethylene glycol monolaurate (PEGML; see, for example, U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly salicylic acid and salicylates, citric acid, and succinic acid. The book Percutaneous Penetration Enhancers (Smith et al., editors, CRC Press, 1995) provides an excellent overview of the field and further background information on several chemical and physical enhancers.

It has long been thought that strong bases, such as NaOH, were not suitable as permeation enhancers because they would damage skin. It has now been discovered that the skin permeability of various drugs could be enhanced without skin damage by exposing the skin to a base or basic solution, in a skin contacting formulation or patch. The desired pH of the solution on the skin can be obtained using a variety of bases or base concentrations. Accordingly, the pH is selected to be low enough so as to not cause skin damage, but high enough to enhance skin permeation to various active agents. As such, in some embodiments, it is important that the amount of base in any patch or formulation is optimized to increase the flux of the drug through the body surface while minimizing any possibility of skin damage. In general, this means that the pH at the body surface in contact with a formulation or drug delivery system may be in the range of approximately pH 8.0 to about pH 13.0, about pH 8.0 to about pH 11.5, about pH 8.5 to about pH 11.5, or about pH 8.5 to about pH 10.5. In some embodiments, the pH is in the range of about pH 9.5 to about pH 11.5, or about pH 10.0 to about pH 11.5.

In one embodiment, the pH at the skin surface is the primary design consideration, i.e., the composition or system is designed to provide the desired pH at the skin surface. Anhydrous formulations and transdermal systems may not have a measurable pH, and the formulation or system can be designed to provide a target pH at the skin surface. Moisture from the body surface can migrate into the formulation or system, dissolve the base and thus release the base into solution, which will then provide the desired target pH at body surface. In those instances, a hydrophilic composition may be desirable. In addition, when using aqueous formulations, the pH of the formulation may change over time after it is applied on the skin. For example, gels, solutions, ointments, etc., may experience a net loss of moisture after being applied to the body surface, i.e., the amount of water lost is greater than the amount of water received from the body surface. In that case, the pH of the formulation may be different than its pH when manufactured. This problem can be easily remedied by designing the aqueous formulations to provide a target pH at the body surface.

In other embodiments, the pH of the formulation or the drug composition contained within a delivery system will be in the range of approximately pH 8.0 to about pH 13.0, about pH 8.0 to about pH 11.5, about pH 8.5 to about pH 11.5, or about pH 8.5 to about pH 10.5. In some embodiments, the pH will be in the range of about pH 9.5 to about pH 11.5, or about pH 10.0 to about pH 11.5. In one embodiment the pH of the formulation is higher than the pH at the body surface. For example, if an aqueous formulation is used, moisture from the body surface can dilute the formulation, and thus provide for a different pH at the body surface, which will typically be lower than that of the formulation itself.

In one embodiment, the body surface is exposed to a base or basic solution for a sufficient period of time so as to provide a high pH at the skin surface, thus creating channels in the skin or mucosa for the drug to go through. It is expected that drug flux is proportional to the strength of the solution and the duration of exposure. However, it is desirable to balance the maximization of drug flux with the minimization of skin damage. This can be done in numerous ways. For example, the skin damage may be minimized by selecting a lower pH within the 8.0 to 13.0 range, by exposing the skin to the formulation or system for a shorter period of time, or by including at least one irritation-mitigating additive. Alternatively, the patient can be advised to change the location of application with each subsequent administration.

While certain amounts are set forth below, it is understood that, for all the inorganic and organic bases described herein, the optimum amount of any such base will depend on the strength or weakness of the base and its molecular weight, and other factors such as the number of ionizable sites in the active agent being administered and whether there are any acidic species present in the formulation or patch. One skilled in the art may readily determine the optimum amount for any particular base such that the degree of enhancement is optimized while the possibility of damage to the body surface is eliminated or at least substantially minimized.

Exemplary inorganic bases are inorganic hydroxides, inorganic oxides, inorganic salts of weak acids, and combinations thereof. Some inorganic bases are those whose aqueous solutions have a high pH and are acceptable as food or pharmaceutical additives. Examples of such inorganic bases include ammonium hydroxide, sodium hydroxide, potassium hydroxide, calcium hydroxide, magnesium hydroxide, magnesium oxide, calcium oxide, Ca(OH)2, sodium acetate, sodium borate, sodium metaborate, sodium carbonate, sodium bicarbonate, sodium phosphate, potassium carbonate, potassium bicarbonate, potassium citrate, potassium acetate, potassium phosphate and ammonium phosphate and combinations thereof.

Inorganic hydroxides include, for example, ammonium hydroxide, alkali metal hydroxide and alkaline earth metal hydroxides, and mixtures thereof. Some inorganic hydroxides include ammonium hydroxide; monovalent alkali metal hydroxides such as sodium hydroxide and potassium hydroxide; divalent alkali earth metal hydroxides such as calcium hydroxide and magnesium hydroxide; and combinations thereof.

The amount of inorganic hydroxide included in the compositions and systems described herein will typically represent about 0.3-7.0 W/V %, about 0.5-4.0 W/V %, about 0.5-3.0 W/V %, or about 0.75-2.0 W/V % of a topically applied formulation or of a drug reservoir of a drug delivery system, or patch.

Inorganic oxides include, for example, magnesium oxide, calcium oxide, and the like.

The amount of inorganic oxide included in the compositions and systems described herein may be substantially higher than the numbers set forth above for the inorganic hydroxide, and may be as high as 20 wt %, in some cases as high as 25 wt % or higher but will generally be in the range of about 2-20 wt %. These amounts may be adjusted to take into consideration the presence of any base-neutralizable species.

Inorganic salts of weak acids include, ammonium phosphate (dibasic); alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic); alkaline earth metal salts of weak acids such as magnesium phosphate and calcium phosphate; and the like, and combinations thereof.

Organic bases suitable for use are compounds having an amino group, amido group, an oxime, a cyano group, an aromatic or non-aromatic nitrogen-containing heterocycle, a urea group, and combinations thereof. More specifically, examples of suitable organic bases are nitrogenous bases, which include, but are not limited to, primary amines, secondary amines, tertiary amines, amidines, guanidines, hydroxylamines, cyano guanidines, cyanoamidines, oximes, cyano (—CN) containing groups, aromatic and non-aromatic nitrogen-containing heterocycles, urea, and mixtures thereof. In some embodiments, the organic bases are primary amines, secondary amines, tertiary amines, aromatic and non-aromatic nitrogen-containing heterocycles, and mixtures thereof.

For all permeation-enhancing bases herein, the optimum amount of any particular agent will depend on the strength or weakness of the base, the molecular weight of the base, and other factors such as the number of ionizable sites in the drug administered and any other acidic species in the formulation or patch. One skilled in the art may readily determine the optimum amount for any particular agent by ensuring that a formulation is effective to provide a pH at the skin surface, upon application of the formulation, in the range of about pH 7.5 to about pH 13.0, about pH 8.0 to about pH 11.5, or about pH 8.5 to about pH 10.5. In some embodiments, the pH will be in the range of about pH 9.5 to about pH 11.5, or about pH 10.0 to about pH 11.5. This in turn ensures that the degree of treatment is maximized while the possibility of damage to the body surface is eliminated or at least substantially minimized.

Additional permeation enhancers will be known to those of ordinary skill in the art of topical drug delivery, and/or are described in the pertinent texts and literature. See, for example, Percutaneous Penetration Enhancers Drug Penetration Into/Through the Skin, Dragicevic & Maibach. (Springer Link, 2017).

### Other Active Agents

In one embodiment, the methods involve the administration of one or more additional drugs for the improvement of sensory function of the anogenitals. In some embodiments, the second therapeutic agent is an antioxidant, anti-inflammatory agent, antimicrobial including antibacterial, antihistamine, mast cell stabilizer, antiviral and antifungal agents, anti-angiogenic agent, anti-apoptotic agent, lubricant, vasodilator, and/or secretagogue.

Inflammation is induced by the process of leukocyte adhesion and neovascularization. Therefore, other anti-inflammatory agents may be administered in combination, prior to, after, or concomitantly with the compounds described herein. The anti-inflammatory agents can be chosen from corticosteroid related drugs including but not limited to dexamethasone, fluoromethalone, medrysone, betamethasone, triamcinolone, triamcinolone acetonide, prednisone, prednisolone, hydrocortisone, rimexolone, and pharmaceutically acceptable salts thereof, prednicarbate, deflazacort, halomethasone, tixocortol, prednylidene, prednival, paramethasone, methylprednisolone, meprednisone, mazipredone, isoflupredone, halopredone acetate, halcinonide, formocortal, flurandrenolide, fluprednisolone, fluprednidine acetate, fluperolone acetate, fluocortolone, fluocortin butyl, fluocinonide, fluocinolone acetonide, flunisolide, flumethasone, fludrocortisone, fluclorinide, enoxolone, difluprednate, diflucortolone, diflorasone diacetate, desoximetasone (desoxymethasone), desonide, descinolone, cortivazol, corticosterone, cortisone, cloprednol, clocortolone, clobetasone, clobetasol, chloroprednisone, cafestol, budesonide, beclomethasone, amcinonide, allopregnane acetonide, alclometasone, 21-acetoxypregnenolone, tralonide, diflorasone acetate, deacylcortivazol, RU-26988, budesonide, deacylcortivazol, and the like. Additionally anti-inflammatory agents include 5-aminosalicylate (5-ASA) compounds, such as sulfasalzine (Azulfidine), osalazine (Dipentum), and mesalamine (examples include Pentasa, Asacol, Dipentum, Colazal, Rowasa enema, and Canasa suppository). Similarly, the anti-inflammatory agents can be chosen from cyclosporine related drugs (for example, calcineurin antagonist) including but not limited to members of the cyclosporine family, and other related calcineurin antagonists including sirolimus, tacorlimus and pimecrolimus. Alternatively, the anti-inflammatory agents can be chosen from the group of NSAIDs including but not limited to acetaminophen, acemetacin, aceclofenac, alminoprofen, amfenac, bendazac, benoxaprofen, bromfenac, bucloxic acid, butibufen, carprofen, celecoxib, cinmetacin, clopirac, diclofenac, etodolac, etoricoxib, felbinac, fenclozic acid, fenbufen, fenoprofen, fentiazac, flunoxaprofen, flurbiprofen, ibufenac, ibuprofen, indomethacin, isofezolac, isoxicam, isoxepac, indoprofen, ketoprofen, lonazolac, loxoprofen, mefenamic acid, meclofenamic acid, meloxicam, metiazinic acid, mofezolac, miroprofen, naproxen, niflumic, oxaprozin, pirozolac, pirprofen, pranoprofen, protizinic acid, rofecoxib, salicylic acid and its derivatives (i.e., for example, aspirin), sulindac, suprofen, suxibuzone, triaprofenic acid, tolmetin, valdecoxib, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, mesalamine, prodrugs thereof, and the like. Additionally, immunomodulators such as 6-mercaptopurine (6-MP), azathioprine (Imuran), methotrexate (Rheumatrex, Trexall), infliximab (Remicade), and adalimumab (Humira) may be used.

A class of therapeutic agents which may be useful to administer in combination, prior to, after, or concomitantly with the compounds described herein is antihistamines, including alkylamine, ethanolamine and phenothiazine classes, such as, for example, chlorpheniramine maleate, chlorphenamiramine tannate, diphenhydramine hydrochloride, promethazine hydrochloride, acrivastine, azatadine maleate, azelastine hydrochloride, brompheniramine maleate, carbinoxamine maleate, cetirizine hydrochloride, clemastine fumarate, cyproheptadine hydrochloride, desloratadine, dexbrompheniramine maleate, dexchlorpheniramine maleate, dimenhydriunate, diphenhydramine hydrochloride, emedastine difumarate, fexofenadine hydrochloride, hydroxyzine hydrochloride, ketotifen fumarate, loratadine, meclizine hydrochloride, olopatadine hydrochloride, phenindamine tartrate, quetiapine, tripelennamine citrate, tripelennamine hydrochloride, and triprolidine hydrochloride. In some embodiments, the formulations administered nasally or to the eye include one or more antihistamines.

A class of therapeutic agents which may be useful to administer in combination, prior to, after, or concomitantly with the compounds described herein is mast cell stabilizers such as cromolyn sodium and nedocromil.

Oxidative stress may be induced in cells with impaired autoregulatory and ischemic processes. Therefore, anti-oxidants may be useful to administer in combination, prior to, after, or concomitantly with the compounds described herein. Examples of suitable anti-oxidants useful in the methods described herein include, but are not limited to, ascorbic acid, tocopherols, tocotrienols, carotinoids, glutathione, alpha-lipoic acid, ubiquinols, bioflavonoids, carnitine, and superoxide dismutase mimetics, such as, for example, 2,2,6,6-tetramethyl-1-piperidinyloxy (TEMPO), DOXYL, PROXYL nitroxide compounds; 4-hydroxy-2,2,6,6-tetramethyl-1-piperidinyloxy (Tempol), M-40401, M-40403, M-40407, M-40419, M-40484, M-40587, M-40588, and the like.

In some embodiments, methods are provided wherein anti-apoptotic therapeutic agents may be administered in combination, prior to, after, or concomitantly with compounds described herein. Examples of suitable anti-apoptotic agents are, for example, inhibitors of caspases, cathepsins, and TNF-α.

Another class of therapeutic agents which may be useful to administer in combination, prior to, after, or concomitantly with the compounds described herein are antimicrobial agents. Suitable antimicrobial compounds, include, but are not limited to, penicillins, such as, for example, amoxicillin, ampicillin, azlocillin, carbenicillin, cloxacillin, dicloxacillin, flucloxacillin, mezlocillin, nafcillin, penicillin, piperacillin, ticarcillin, and the like; beta-lactamase inhibitors; carbapenems, such as, for example, ertapenem, imipenem, meropenem, and the like; cephalosporins, such as, for example, cefaclor, cefamandole, cefoxitin, cefprozil, cefuroxime, cefixime, cefdinir, cefditoren, cefoperazone, cefotaxime, cefpodoxime, cefadroxil, ceftazidime, ceftibuten, ceftizoxime, ceffiriaxone, cefazolin, cefixime, cephalexin, cefepime, and the like; quinolones, such as, for example, ciprofloxacin, enoxacin, gatifloxacin, levofloxacin, lomefloxacin, morifloxacin, norfloxacin, ofloxacin, trovafloxacin, and the like; macrolides, such as, for example, azithromycin, clarithromycin, dirithromycin, erythromycin, milbemycin, troleandomycin, and the like; monbactams, and the like; tetracyclins, such as, for example, demeclocyclin, doxycycline, minocycline, oxytetracyclin, tetracycline, and the like; aminoglycosides, such as, for example, amikacin, gentamicin, kanamycin, neomycin, netilmicin, paromomycin, streptomycin, tobramycin, and the like; carbacephem, such as, for example, loracarbef, and the like; streptogramins; sulfonamides, such as, for example, mefanide, prontosil, sulfacetamide, sulfamethizole, sulfanilamide, sulfasalazine, sulfisoxazole, trimethoprim, trimethoprim-sultamethoxazole, and the like; other antimicrobials such as metronidazole; and the combination drugs such as for example, sulfamethoxazole and trimethoprim, and the like.

Other antimicrobial agents include the class of antiviral agents. Antiviral agents include but are not limited to therapeutic agents such as entry inhibitors, reverse transcriptase inhibitors, nucleoside or nucleotide analogs, protease inhibitors, and inhibitors of viral release from host cells. Some illustrative therapeutic agents of this group, include, but are not limited to abacavir, acyclovir, adefovir, amantadine, amprenavir, arbidol, atazanavir, atripla, brivudine, cidofovir, combivir, darunavir, delavirdine, didanosine, docosanol, edoxudine, efavirenz, emtricitabine, enfuvirtide, entecavir, famciclovir, fomivirsen, foscarnet, fosfonet, ganciclovir, gardasil, ibacitabine, immunovir, idoxuridine, imiquimod, indinavir, inosine, interferon type III, interferon type II, interferon type I, interferon, lamivudine, lopinavir, loviride, maraviroc, moroxydine, nelfinavir, neviapine, nexavir, oseltamivir, penciclovir, peramivir, pleconaril, podophyllotoxin, raltegravir, ribavirin, rimantadine, ritonavir, saquinavir, stavudine, tenofovir, tenofovir disoproxil, tipranavir, trifluridine, trizivir, tromantadine, truvada, valaciclovir, valganciclovir, vicriviroc, vidarabine, viramidine, zalcitabine, zanamivir, zidovudine, and the like.

In some of the embodiments, the formulations administered to the skin comprise one or more antimicrobial or antibiotic agents.

Secretagogues may also be administered in combination, prior to, concomitantly with, or after administration pirenzepine. Increasing mucin or other fluid production in the eye may be beneficial. Examples include but are not limited to Diquafasol, Rebamipide, and Eicosanoid 15-(S)-HETE.

A class of therapeutic agents which may be useful to administer in combination, prior to, after, or concomitantly with pirenzepine are vasodilators that help relax blood vessels to improve blood flow, including phosphodiesterase inhibitors (papaverine, pentoxifylline, and amrinone), local anesthetics (lidocaine), calcium channel blockers (nicardipine, verapamil, nifedipine, and magnesium sulfate), direct vasodilators (sodium nitroprusside, prostaglandin E1, nitroglycerin, and hydralazine), and alpha antagonists (phentolamine and chlorpromazine).

Alprostadil is a vasodilator that treats ED effectively for many men. Alprostadil is a synthetic analog of prostaglandin E1 (PGE1) and shows a multifariousness of pharmacologic actions. Alprostadil binds as an agonist to prostaglandin receptors, for example, EP2 which in turns activates adenylate cyclase leading to accumulation of 3′5′-cAMP (cyclic adenosine monophosphate).

Non-limiting examples of phosphodiesterase type 5 inhibitors include, but are not limited to, avanafil, lodenafil, mirodenafil (pKa of 6.0), sildenafil (or analogs thereof, for example, actetildenafil, hydroxyacetildenafil, or dimethylsildenafil), tadalafil (pKa of 18), vardenafil (pKas of 3.4, 6.7, 8.8, and 14), udenafil (pKa of 10.53), acetildenafil, or thiomethisosildenafil.

Many phosphodiesterase type 5 inhibitors are readily commercially available. In some cases, the phosphodiesterase type 5 inhibitor may be obtained as a racemic mixture, for example, of tadalafil (for example, (R,R)-tadalafil, (R,S)-tadalafil, (S,R)-tadalafil, and (S,S)-tadalafil). However, in other cases, one of the enantiomers may be present in an amount greater than the other. For example, at least about 60%, at least about 70%, at least about 80%, at least about 90%, or at least about 95% of the phosphodiesterase type 5 inhibitor within the composition may be present as one of the enantiomers. Techniques for preparing or separating racemic phosphodiesterase type 5 inhibitors are known; see, for example, Gao, et al., “Chiral Separation of Two Pairs of Enantiomers of Tadalafil by High-Performance Liquid Chromatography,” J. Chromatogr. Sci., 45:540-543, 2007.

The composition may also comprise a nitric oxide donor in some embodiments, for example, L-arginine and/or L-arginine hydrochloride. In some cases, such a nitric oxide donor may be used to increase localized blood flow at the site where the composition is applied, which may enhance delivery of the pharmaceutical agent. The nitric oxide donor may be present at any suitable concentration within the composition. For instance, in some cases, the nitric oxide donor is present at a concentration of at least about 1%, at least about 2%, at least about 3%, at least about 4%, at least about 5%, at least about 6%, at least about 7%, at least about 7.5%, at least about 8%, at least about 9%, or at least about 10% by weight of the composition. In some cases, one or more nitric oxide donors (for example, 2, 3, 4, 5, 6, 7, 8, 9, 10, etc. nitric oxide donors) may be used. In some cases, there may be no more than 3, 5, 7, or 10 nitric oxide donors present within the composition.

A “nitric oxide donor,” as used herein, is a compound that can release nitric oxide and/or chemically transfer the nitric oxide moiety to another molecule, directly or indirectly, for example, through a biological process. The nitric oxide donor may release nitric oxide into the skin, and/or tissues such as muscles and/or elements of the circulatory system in close proximity to the surface of the skin. Non-limiting examples of nitric oxide donors include arginine (for example, L-arginine and/or D-arginine), arginine derivatives (for example, L-arginine hydrochloride and/or D-arginine hydrochloride), nitroglycerin, polysaccharide-bound nitric oxide-nucleophile adducts, N-nitroso-N-substituted hydroxylamines, 1,3-(nitrooxymethyl)phenyl-2-hydroxybenzoate, etc., and/or any combination of these and/or other compounds.

Besides L-arginine and L-arginine hydrochloride, other non-limiting examples of nitric oxide donors include D,L-arginine, D-arginine, or alkyl (for example, ethyl, methyl, propyl, isopropyl, butyl, isobutyl, tert-butyl, etc.) esters of L-arginine and/or D-arginine (for example, a methyl ester, an ethyl ester, a propyl ester, a butyl ester, etc.) and/or salts thereof, as well as other derivatives of arginine and other nitric oxide donors. For instance, non-limiting examples of pharmaceutically acceptable salts include hydrochloride, glutamate, butyrate, or glycolate (for example, resulting in L-arginine glutamate, L-arginine butyrate, L-arginine glycolate, D-arginine hydrochloride, D-arginine glutamate, etc.). Still other examples of nitric oxide donors include L-arginine-based compounds such as, but not limited to, L-homoarginine, N-hydroxy-L-arginine, nitrosylated L-arginine, nitrosylated L-arginine, nitrosylated N-hydroxy-L-arginine, nitrosylated N-hydroxy-L-arginine, citrulline, ornithine, linsidomine, nipride, glutamine, etc., and salts thereof (for example, hydrochloride, glutamate, butyrate, glycolate, etc.), and/or any combination of these and/or other compounds. Still other non-limiting examples of nitric oxide donors include S-nitrosothiols, nitrites, 2-hydroxy-2-nitrosohydrazines, or substrates of various forms of nitric oxide synthase. In some cases, the nitric oxide donor may be a compound that stimulates endogenous production of nitric oxide in vivo. Examples of such compounds include, but are not limited to, L-arginine, substrates of various forms of nitric oxide synthase, certain cytokines, adenosine, bradykinin, calreticulin, bisacodyl, phenolphthalein, OH-arginine, or endothelein, and/or any combination of these and/or other compounds.

Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, purite, peroxides, perborates, and combinations thereof.

The formulation may also contain an aesthetic agent. Examples of aesthetic agents include fragrances, pigments, colorants, essential oils, skin sensates and astringents. Suitable aesthetic agents include clove oil, menthol, camphor, eucalyptus oil, eugenol, methyl lactate, bisabolol, witch hazel distillate and green tea extract.

Fragrances are aromatic substances which can impart an aesthetically pleasing aroma. Typical fragrances include aromatic materials extracted from botanical sources (i.e., rose petals, gardenia blossoms, jasmine flowers, etc.) which can be used alone or in any combination to create essential oils. Alternatively, alcoholic extracts may be prepared for compounding fragrances. However, due to the relatively high costs of obtaining fragrances from natural substances, the modern trend is to use synthetically prepared fragrances, particularly in high-volume products. One or more fragrances can optionally be included in the sunscreen composition in an amount ranging from about 0.001 to about 5 weight percent, p or about 0.01 to about 0.5 percent by weight. Additional preservatives may also be used if desired and include well known preservative compositions such as benzyl alcohol, phenyl ethyl alcohol and benzoic acid, diazolydinyl, urea, chlorphenesin, iodopropynyl and butyl carbamate, among others.

In one embodiment, the formulation is a formulation as described in WIPO Patent Application WO/2020/198252, Topical Formulations for the Treatment of Peripheral Neuropathy, such as the topical formulations of pirenzepine or a salt thereof, in combination with DMSO and a polyether surfactant, for example polyalkylene glycol alkyl ether.

### Composition Forms

The active agent can be administered to a patient in any pharmaceutically acceptable and effective form, for example topically via creams, gels, ointments, emulsions, solutions, lotions, suspensions, tinctures, pastes, foams, aerosols, irrigations, sprays, suppositories, or any other formulations or pharmaceutical compositions suitable for topical administration, and the like. Formulations for topical use of the pharmaceutical compositions can be provided as a topical composition wherein the pharmacologically active ingredients are mixed with excipients to form a semisolid consistency. Examples of such topical pharmaceutical compositions include, but are not limited to, a gel, cream, lotion, suspension, emulsion, ointment, foam, paste and the like. Alternatively, the topical pharmaceutical compositions can be formulated in a semi-liquid formulation. Examples of such topical pharmaceutical compositions include, but are not limited to, a topical solution, spray, mist, drops and the like. Alternatively, the topical pharmaceutical compositions can be formulated in a dry powder form. The pharmaceutical compositions can also be administered by a transdermal patch.

Ointments, as is well known in the art of pharmaceutical formulation, are semi-solid preparations that are typically based on petrolatum or other petroleum derivatives. As an ointment, the composition has a consistency suitable for uniform dermal application. Additionally, the ointment may be substantially viscous to remain in contact with the skin regardless of perspiration, excess moisture, or environmental conditions. The specific ointment base to be used, as will be appreciated by those skilled in the art, is one that will provide for optimum drug delivery, and, will provide for other desired characteristics as well, for example, emolliency or the like. As with other carriers or vehicles, an ointment base should be inert, stable, nonirritating and nonsensitizing. As explained in Remington: The Science and Practice of Pharmacy, 19^{th} Ed. (Easton, Pa.: Mack Publishing Co., 1995), at pages 1399-1404, ointment bases may be grouped in four classes: oleaginous bases; emulsifiable-bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Some water-soluble ointment bases are prepared from polyethylene glycols of varying molecular weight; again, see Remington: The Science and Practice of Pharmacy for further information.

Creams, as also well known in the art, are viscous liquids or semi-solid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

Gels are semi-solid, suspension-type systems and are well known in the art. Gel forming agent for use herein can be any gelling agent typically used in the pharmaceutical art for topical semi solid dosage forms. Single-phase gels contain organic macromolecules distributed substantially uniformly throughout the carrier liquid, which is typically aqueous, but also can contain an alcohol and optionally an oil. To prepare a uniform gel, dispersing agents such as alcohol or glycerin can be added, or the gelling agent can be dispersed by tritration, mechanical mixing or stirring, or combinations thereof. The number of gelling agents varies widely and will ordinarily range from about 0.1% to about 2.0% by weight, based on the total weight of the composition. The gel forming agent also works by the principle of copolymerization. Under alkaline pH, carbomer in presence of water undergoes cross linking and forms a gel like structure. The degree of polymerization is dependent upon the pH. At a threshold pH, the viscosities achieved by the polymer grade are the maximum.

Lotions are preparations to be applied to the skin surface without friction and are typically semi-liquid preparations in which solid particles, including the active agent, are present in a water or alcohol base. Lotions are usually suspensions of solids, and for the present purpose, comprise a liquid oily emulsion of the oil-in-water type. Lotions may be desirable formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, for example, methylcellulose, sodium carboxymethyl-cellulose, or the like.

Pastes are semi-solid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Plasters are comprised of a pasty mixture that is spread on the body, either directly or after being saturated into a base material such as cloth. Medications, including pharmacologically active bases, may be dissolved or dispersed within the plaster to make a medicated plaster.

Bioadhesives are preparations that adhere to surfaces of body tissues. Polymeric bioadhesive formulations are well known in the art; see, for example, Heller et al., “Biodegradable polymers as drug delivery systems,” in Chasin, M. and Langer, R., eds.: Dekker, N.Y., pp. 121-161 (1990); and U.S. Pat. No. 6,201,065. Suitable non-polymeric bioadhesives are also known in the art, including certain fatty acid esters (U.S. Pat. No. 6,228,383).

A pharmaceutical composition for transdermal administration may be provided as, for example, a hydrogel comprising pirenzepine incorporated into an adhesive patch composition. Intended to remain in intimate contact with a subject's epidermis for a prolonged period of time. An exemplary adhesive patch composition can comprise a monolithic layer produced by mixing a muscarinic acetylcholine receptor antagonist(s) with a silicone-type adhesive or alternatively an acrylate-vinyl acetate adhesive in a solvent exemplified by methylene chloride, ethyl acetate, isopropyl myristate, and propylene glycol. The mixture would then be extruded onto a polyester-backing film to a uniform thickness of about 100 microns or greater with a precision wet-film applicator. The solvent is allowed to evaporate in a drying oven and the resulting “patch” is trimmed to the appropriate size.

Another type of carrier useful to deliver the copper ion treatments to the vagina and rectum is a suppository. Suppositories are commonly used in the vagina and rectum (anus) as a means for dispensing various active ingredients or medicaments. Suppositories are made in various shapes including oviform, globular, conical and bullet shapes, and in various sizes. Suppositories typically weigh in the range of 1 to 5 grams. Suppositories can be solid bodies composed of a mixture of a suitable suppository base material and the active ingredients or medicaments. Alternatively, suppositories can be made with a solid outer wall of suppository base material enclosing non-solid active ingredients or medicaments. The suppository base materials used in suppositories allow them to dissolve or melt when exposed to the moisture (body fluid) or heat (body temperature) found in the vagina or rectum (rectal or anal canal), thereby releasing the active ingredients or medicaments into the vagina or rectum. Suitable suppository base materials include oleaginous (fatty) base materials, including cocoa butter, theobroma oil and synthetic triglycerides, or water soluble or miscible base materials, including glycerinated gelatin and polyethylene glycol (PEG) polymers. It is preferred that the base materials be non-toxic, non-irritating, inert, and biocompatible. Suppositories suitable for use in an aspect of the present disclosure can be prepared in various ways according to conventional methods for preparing suppositories including compression molding and fusion molding. The suppositories may be inserted in the vagina and rectum using the fingers, or the suppositories may be provided with applicators to facilitate insertion thereof in the vagina and rectum.

Another type of carrier that can be used to deliver treatments to the vagina is a tampon. The tampon used can be a commercially available tampon or one similar thereto. The tampon can be one having an applicator including a barrel containing the absorbent tampon body and a plunger slidable within the barrel to dispose or eject the absorbent tampon body from an open forward end of the barrel once the forward end has been introduced in the vagina an appropriate distance in a commonly known manner of tampon use. Another suitable tampon can be one without an applicator, i.e., a digital tampon, where the absorbent tampon body is inserted in the vagina by pushing it with the fingers.

Another type of carrier that can be used to deliver the treatments to anatomical tissue is a body wipe. The body wipe comprises a thin sheet of material with the treatment which can be overlappingly folded to be removed from a package. Upon removal from the package, the body wipe can be unfolded to its full size, which is substantially larger than its size in the folded condition, and can be used to wipe anatomical tissue to be treated causing the copper ion treatment to be transferred to the anatomical tissue.

Another type of carrier for the treatments is a wound dressing, such as a band aid, gauze pad or similar device. Such carriers can be selected from products that are commercially available for removable application to the skin to temporarily cover and protect an affected area of the skin.

A further type of carrier for the treatments is a skin patch, such as a dermal patch or a transdermal patch. The skin patch has a drug delivery surface containing the treatment surrounded by an adhesive border. The patch is applied to the skin and is left in place for a period of time with the drug delivery surface in contact with the skin, causing the active ingredients to diffuse through the skin where they can act locally or penetrate the capillaries for broader systemic effects. Examples of suitable transdermal patches are the transdermal and microneedle 3M Drug Delivery Systems manufactured by 3M corporation.

### Methods of Treatment Using Topically Formulated Pirenzepine

In some embodiments, compounds are therapeutically and/or prophylactically useful for treating sensory diseases or conditions of the anogenitals of a subject. Accordingly, in one aspect, a method is provided for treatment a sexual sensory condition or disorder in a subject comprising topically administering to the anogenitals of said subject a formulation comprising pirenzepine or a pharmaceutically acceptable salt or ester thereof, and a pharmaceutically acceptable excipient.

The benefits of topical administration include localized delivery of the therapeutic agent and minimal systemic side effects due to low systemic bioavailability. For example, topical formulations may be administered directly to the anogenital region of a subject including the skin, and the vaginal mucosa or anal mucosa. The methods of topical delivery are particularly well suited for localized administration of the formulation. Suitable formulations and additional carriers are discussed herein and, additionally, described in Remington “The Science and Practice of Pharmacy” (20^{th} Ed., Lippincott Williams & Wilkins, Baltimore Md.), the teachings of which are incorporated by reference in their entirety herein.

The compositions may be delivered with a pharmacokinetic profile that results in the delivery of an effective dose of pirenzepine. The actual effective amounts of drug can vary according to the specific drug or combination thereof being utilized, the particular composition formulated, the mode of administration, and the age, weight, condition of the patient, and severity of the symptoms or condition being treated. Dosages for a particular patient can be determined by one of ordinary skill in the art using conventional considerations, (for example, by means of an appropriate, conventional pharmacological protocol).

In other embodiments, provided herein is a method for improving the sensitivity of the skin in the anogenital region comprising applying to the skin an effective amount of a topical composition comprising pirenzepine.

In some embodiments, provided herein is a method for improving the symptoms of neuropathy comprising applying to the skin in the anogenital region an effective amount of a topical composition comprising pirenzepine. In a preferred embodiment, the neuropathy is diabetic neuropathy.

In certain embodiments, the topical compositions provided herein are intended for topical, non-invasive, application to the anogenital regions, especially the penis, its entirety, or preferably just the glans of the penis. In addition, the composition may be applied to the shaft of the penis.

### Administration

The method of delivery of the pharmaceutically active composition may vary, and in some embodiments involves application of a formulation to an anogenital region affected with sensory disruption. In some embodiments, the formulation is topically applied to anogenital region, including the skin, vaginal mucosa, or anal mucosa. A gel, cream, ointment, paste, plaster, or lotion may be spread on the affected area of skin and gently rubbed in. Similarly, a polymeric or other bioadhesive formulation may be spread or dabbed on the affected area of skin. A solution may be applied in the same ways, but more typically will be applied with a dropper, swab, or the like, and carefully applied to the affected area of skin. Petrolatum may be spread on the skin surrounding the affected area of skin to protect it from possible irritation during treatment.

The dosing regimen will depend on a number of factors that may readily be determined, such as the size of the affected area, the severity of the affected area, and the responsiveness of the affected area to treatment, but will normally be one or more doses per day, with a course of treatment lasting from several days to several months, or until a cure is effected or a significant diminution in the size and/or severity of the sensory disruption is achieved. One of ordinary skill may readily-determine optimum dosages, dosing methodologies, and repetition rates. In general, it is contemplated that the formulation will be applied one to four times daily. With a skin patch, suppository or other device, the device is generally maintained in place on the body surface throughout a drug delivery period, typically in the range of 8 to 72 hours, and replaced as necessary.

In some embodiments, pirenzepine is present in an amount sufficient to exert a therapeutic effect by an average of at least about 5, 10, 15, 20, 25, 30, 40, 50, 60, 70, 80, 90, more than 90%, or substantially eliminate all symptoms.

In some embodiments, an effective amount of pirenzepine is a dose of about 1×10⁻¹¹, 1×10⁻¹⁰, 1×10⁻⁹, 1×10⁻⁸, 1×10⁻⁷, 1×10⁻⁶, 1×10⁻⁵, 1×10⁻⁴, 1×10⁻³, 1×10⁻², 1×10⁻¹, 1, 1×10¹ or 1×10² grams.

A method for treatment of sensory disorders comprises administration of the formulations in topical form.

The total daily doses of the medicaments contemplated for use, and consequently the concentrations by weight of the medicaments in the respective compositions, may vary widely, but are within the typical skill of the routine practitioner. The specific therapeutically effective dose level for any particular patient and medicament will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific medicament employed; the duration of the treatment; drugs used in combination or coincidental with the specific compound employed; and like factors well known in the medical arts. For example, it is well within the skill of the art to start doses at levels lower than required to achieve the desired therapeutic effect and to gradually increase the dosage until the desired effect is achieved.

In some embodiments, the composition is administered in a single dose. A single dose of pirenzepine may also be used when it is co-administered with another substance (for example, an analgesic) for treatment of an acute condition.

In some embodiments, pirenzepine (by itself or in combination with other drugs) is administered in multiple doses. Dosing may be about once, twice, three times, four times, five times, six times, seven times, eight times, nine times, ten times or more than ten times per day. Dosing may be about once a year, twice a year, every six months, every 4 months, every 3 months, every 60 days, once a month, once every two weeks, once a week, or once every other day. In one embodiment the drug is an analgesic. In another embodiment pirenzepine and another therapeutic substance are administered together about once per day to about 10 times per day. In another embodiment, an additional therapeutic substance is administered concurrent with, prior to, or subsequent to administering pirenzepine. In another embodiment the administration of pirenzepine and another therapeutic substance continues for less than about 7 days. In yet another embodiment the co-administration continues for more than about 6, 10, 14, a month, two months, six months, or one year. In some cases, co-administered dosing is maintained as long as necessary, for example, dosing for chronic or degenerative complications.

Administration of the compositions may continue as long as necessary. In some embodiments, a composition is administered for more than 1, 2, 3, 4, 5, 6, 7, 14, or 28 days. In some embodiments, a composition is administered for less than 28, 14, 7, 6, 5, 4, 3, 2, or 1 day. In some embodiments, a composition is administered chronically on an ongoing basis, for example, for the treatment of chronic pain.

Dosing for the formulation in the methods described herein may be found by routine experimentation. The daily dose can range from about 1×10⁻⁷ g to 5000 mg. Daily dose range may depend on the form of pirenzepine for example, the esters or salts used, and/or route of administration, as described herein. For example, for systemic administration, typical daily dose ranges are, for example, about 1-5000 mg, or about 1-3000 mg, or about 1-2000 mg, or about 1-1000 mg, or about 1-500 mg, or about 1-100 mg, or about 10-5000 mg, or about 10-3000 mg, or about 10-2000 mg, or about 10-1000 mg, or about 10-500 mg, or about 10-200 mg, or about 10-100 mg, or about 20-2000 mg or about 20-1500 mg or about 20-1000 mg or about 20-500 mg, or about 20-100 mg, or about 50-5000 mg, or about 50-4000 mg, or about 50-3000 mg, or about 50-2000 mg, or about 50-1000 mg, or about 50-500 mg, or about 50-100 mg, about 100-5000 mg, or about 100-4000 mg, or about 100-3000 mg, or about 100-2000 mg, or about 100-1000 mg, or about 100-500 mg, or may be within a range defined by any of two of the aforementioned amounts per application. In some embodiments, the daily dose of pirenzepine is about 100, 200, 300, 400, 500, 600, 700, 800, 900, or 1000 mg. In some embodiments, the daily dose of pirenzepine is 0.1 mg. In some embodiments, the daily dose of pirenzepine is 1.0 mg. In some embodiments, the daily dose of pirenzepine is 10 mg. In some embodiments, the daily dose of pirenzepine is 5, 10, 20, 30, 40, 50, 60, 70, 80, 90, or 100 mg. In some embodiments, the daily dose of pirenzepine is 500 mg. In some embodiments, the daily dose of pirenzepine is 1000 mg.

The typical daily dose ranges are, for example, about 1×10-7 g to 5.0 g, or about 1×10⁻⁷ g to 2.5 g, or about 1×10⁻⁷ g to 1.00 g, or about 1×10⁻⁷ g to 0.5 g, or about 1×10⁻⁷ g to 0.25 g, or about 1×10⁻⁷ g to 0.1 g, or about 1×10⁻⁷ g to 0.05 g, or about 1×10⁻⁷ g to 0.025 g, or about 1×10⁻⁷ g to 1×10⁻² g, or about 1×10⁻⁷ g to 5×10⁻³ g, or about 1×10⁻⁷ g to 2.5×10⁻³ g, or about 1×10⁻⁷ g to 1×10⁻³ g, or about 1×10⁻⁷ g to 5×10⁻⁴ g, or about 1×10⁻⁶ g to 5.0 g, or about 1×10⁻⁶ g to 2.5 g, or about 1×10⁻⁶ g to 1 g, or about 1×10⁻⁶ g to 0.5 g, or about 1×10⁻⁶ g to 0.25 g, or about 1×10⁻⁶ g to 0.1 g, or about 1×10⁻⁶ g to 5×10⁻² g, or about 1×10⁻⁶ g to 5×10⁻² g, or about 1×10⁻⁶ g to 2.5×10⁻² g, or about 1×10⁻⁶ g to 1×10⁻² g, or about 1×10⁻⁶ g to 5×10⁻³ g, or about 1×10⁻⁶ g to 2.5×10⁻³ g, or about 1×10⁻⁶ g to 1×10⁻³ g, or about 1×10⁻⁶ g to 5×10⁻⁴ g, or about 1×10⁻⁵ g to 5 g, or about 1×10⁻⁵ g to 2.5 g, or about 1×10⁻⁵ g to 1 g, or about 1×10⁻⁵ g to 0.5 g, or about 1×10⁻⁵ g to 0.25 g, or about 1×10⁻⁵ g to 0.1 g, or about 1×10⁻⁵ g to 0.05 g, or about 1×10⁻⁵ g to 2.5×10⁻² g, or about 1×10⁻⁵ g to 1×10⁻² g, or about 1×10⁻⁵ g to 5×10⁻³ g, or about 1×10⁻⁵ g to 2.5×10⁻³ g, or about 1×10⁻⁵ g to 1×10⁻³ g, or about 1×10⁻⁵ g to 5×10⁻⁴ g. In some embodiments, the daily dose of pirenzepine is about 1×10⁻⁷, 1×10⁻⁶, 1×10⁻⁵, 1×10⁻⁴, 1×10⁻³ g, 1×10⁻² g, 1×10¹ g, or 1 g. In some embodiments, the daily dose of pirenzepine is 1×10⁻⁷ g. In some embodiments, the daily dose of pirenzepine is 1×10⁻⁵ g. In some embodiments, the daily dose of pirenzepine is 1×10⁻³ g. In some embodiments, the daily dose of pirenzepine is 1×10⁻² g. In some embodiments the individual dose ranges from about 1×10⁻⁷ g to 5.0 g, or about 1×10⁻⁷ g to 2.5 g, or about 1×10⁻⁷ g to 1.00 g, or about 1×10⁻⁷ g to 0.5 g, or about 1×10⁻⁷ g to 0.25 g, or about 1×10⁻⁷ g to 0.1 g, or about 1×10⁻⁷ g to 0.05 g, or about 1×10⁻⁷ g to 0.025 g, or about 1×10⁻⁷ g to 1×10⁻² g, or about 1×10⁻⁷ g to 5×10⁻³ g, or about 1×10⁻⁷ g to 2.5×10⁻³ g, or about 1×10⁻⁷ g to 1×10⁻³ g, or about 1×10⁻⁷ g to 5×10⁻⁴ g, or about 1×10⁻⁶ g to 5.0 g, or about 1×10⁻⁶ g to 2.5 g, or about 1×10⁻⁶ g to 1 g, or about 1×10⁻⁶ g to 0.5 g, or about 1×10⁻⁶ g to 0.25 g, or about 1×10⁻⁶ g to 0.1 g, or about 1×10⁻⁶ g to 5×10⁻² g, or about 1×10⁻⁶ g to 5×10⁻² g, or about 1×10⁻⁶ g to 2.5×10⁻² g, or about 1×10⁻⁶ g to 1×10⁻² g, or about 1×10⁻⁶ g to 5×10⁻³ g, or about 1×10⁻⁶ g to 2.5×10⁻³ g, or about 1×10⁻⁶ g to 1×10⁻³ g, or about 1×10⁻⁶ g to 5×10⁻⁴ g, or about 1×10⁻⁵ g to 5 g, or about 1×10⁻⁵ g to 2.5 g, or about 1×10⁻⁵ g to 1 g, or about 1×10−5 g to 0.5 g, or about 1×10−5 g to 0.25 g, or about 1×10−5 g to 0.1 g, or about 1×10⁻⁵ g to 0.05 g, or about 1×10⁻⁵ g to 2.5×10⁻² g, or about 1×10⁻⁵ g to 1×10⁻² g, or about 1×10⁻⁵ g to 5×10⁻³ g, or about 1×10⁻⁵ g to 2.5×10⁻³ g, or about 1×10⁻⁵ g to 1×10⁻³ g, or about 1×10⁻⁵ g to 5×10⁻⁴ g. In some embodiments, the individual doses as described above, is repeated 1, 2, 3, 4, 5, 6, 7, 8, 9, or 10 times per day.

The amount of administration and the number of administrations of the active ingredient vary according to sex, age and body weight of patient, symptoms to be treated, desirable therapeutic effects, administration routes and period of treatment. For delivery to an adult, the formulations containing the compounds may range in concentration from about 0.0001 to 10.0 W/V %, about 0.005 to 10.0 W/V %, about 0.01 to 10.0 W/V %, about 0.05 to 10.0 W/V %, about 0.1 to 10.0 W/V %, about 0.5 to 10.0 W/V %, about 1.0 to 10.0 W/V %, about 20 to 10.0 W/V %, about 3.0 to 10.0 W/V %, about 4.0 to 10.0 W/V %, or about 5.0 to 10.0 W/V %. One embodiment has a formulation of about 1.0 to 10.0 W/V % of the compounds. One embodiment has a formulation of about 0.01 to 10.0 W/V % of the compounds. One embodiment has a formulation of about 5.0 to 10.0 W/V % of the compounds. The administration may be administered several times a day, one to ten times, one to four times, or once a day.

When used in the above compositions, a therapeutically effective amount of a medicament may be employed in pure form or, where such forms exist, in pharmaceutically acceptable salt, ester or prodrug form.

### EXAMPLES

The following examples illustrate embodiments of the disclosure that are presently best known. However, it is to be understood that the following are only exemplary or illustrative of the application of the principles of the present disclosure. Numerous modifications and alternative compositions, methods, and systems may be devised by those skilled in the art without departing from the spirit and scope of the present disclosure. The appended claims are intended to cover such modifications and arrangements. Thus, while the present disclosure has been described above with particularity, the following examples provide further detail in connection with what are presently deemed to be the most practical and preferred embodiments of the disclosure.

### Example 1

Materials used in preparing the topical sensitization formulation described herein may be made by known methods or are commercially available. It is also possible to make use of variants which are themselves known to those of ordinary skill in this art, but are not mentioned in greater detail. The skilled artisan given the literature and this disclosure is well equipped to prepare the formulations of the instant application.

Representative topical formulations are shown Table 1 and Table 2 below, with the amounts for “broad” and “narrower” ranges.

TABLE 1
Representative Topical Compositions

| | Broader | Narrower |
|---|---|---|
| Component | Range wt. % | Range wt. % |
| Pirenzepine | 0.1-15.0% | 1.0-6.0% |
| Dimethyl Sulfoxide (DMSO) | 5-50% | 10-30% |
| Capric/caprylic triglyceride | 5-50% | 10-30% |
| Laureth-4, brij L4 | 0.1-10.0% | 1.0-6.0% |
| Ethanol | 0-90% | 10-50% |
| Propylene Glycol | 10.0-30.0% | 15.0-25.0% |
| Hydroxypropyl cellulose | 0.1-10.0% | 1.0-6.0% |
| BHT | 0-10% | .05-1% |

TABLE 2
Representative Topical Compositions

| | Broader | Narrower |
|---|---|---|
| Component | Range wt. % | Range wt. % |
| Pirenzepine | 0.1-15.0% | 1.0-6.0% |
| sorbitol solution USP 70% | 1.0-10.0% | 3.0-7.0% |
| isopropyl alcohol USP 70% | 1.0-10.0% | 2.0-6.0% |
| propylene glycol | 10.0-30.0% | 15.0-25.0% |
| butylated hydroxytoluene | 0.1-3.0% | 0.5-1.5% |
| triethanolamine | 0.1-3.0% | 0.5-1.5% |
| benzyl alcohol | 0.1-3.0% | 0.5-1.5% |
| benzyl benzolate | 0.1-3.0% | 0.5-1.5% |
| PEG 40-hydrogenated castor oil | 1.5-5.5% | 2.5-4.5% |
| acrylate/C10-30 alkyl acrylate cross | 0-5-2.0% | 1.0-1.6% |
| polymer | | |
| disodium EDTA | 0.01-0.20% | 0.05-0.15% |
| water | 40.0-60.0% | 50.0-56.0% |

Use of the Topical solution as that described in Example 1 had the following effects in subjects:

### Example 2

Subject 1 was a 44-year-old man of Asian descent suffering intermittently from the effects of Guillain-Barré Syndrome (GBS) for some years, primarily affecting his mobility. Over 15 years, he had noticed a progression of diminished sensitivity and delayed ejaculation during sex, with an average ejaculation time of 40-50 minutes. A daily application of a formulation of 4% pirenzepine (as described in Example 1) was applied to the entire penis including the head (glans), shaft and scrotum of the penis for 2 weeks, subject noticed a shorter interval to ejaculation of an average of 30 minutes. His orgasm was noticeably more intense.

### Example 3

Subject 2 was a 68-year-old subject. Subject applied a topical formulation as that described in Example 1 to the head (glans) and shaft of the penis once a day in the late evening for 8 weeks. After two weeks the subject noticed a significant and qualitatively improved positive sensory difference during intercourse. Subject did not believe there had been decline in sensitivity, but after 3 weeks treatment discovered there had been significant improvement in sensitivity and intensity, rapidity, urgency, and duration of response to baseline levels of stimulation. No negative side effects were detected, and sensation continued to improve after 4 weeks. Subject remarked that he didn't realize that he'd lost so much sensation until it was restored. Subject had been using tadalafil 10 mg prior to intercourse, and reduced dose to 5 mg as a result of improvement in sensitivity, sustainment and hardness.

### Example 4

Subject 3 was a non-diabetic Asian male 49 years old in good health but with difficulty for approximately 3 years in reaching ejaculation, progressively getting worse. Subject 3 used the provided topical medication inconsistently but on average once a week for three months. During that time subject saw a huge improvement in the ability to reach climax. There was not a single instance since starting treatment that the subject was unable to climax. Subject 3 reported increased occurrences of awakening to a morning erection, and increased sexual sensitivity.

### Example 5

Subject 4 was a 61 year old, male Caucasian diagnosed with Type 2 Diabetes in July 2010. Subject reported a decreasing sexual response over the years, with function occasionally treated with Cialis, but due to side effects, that treatment was discontinued. The subject started application of topical pirenzepine on penis and after two days, there was an increase of sexual response and function. Subject 4 reported that he had nocturnal erections typically for the three days after application, which was applied typically twice weekly. The subject experienced a noticeable sensation after the application around the scrotum where the penis lies. The subject reported functional improvement in the ability to achieve an erection and penetrative intercourse. Ejaculation was similar to patient's experience with Cialis, but without a headache.

It is noted in the above examples that various skin contact areas, analgesic system contact times, methods of treating specific conditions, etc., are provided for exemplary purposes only. Thus, while methods and compositions have been described with reference to certain embodiments, those skilled in the art will appreciate that various modifications, changes, omissions, and substitutions can be made without departing from the spirit of the disclosure.

What is claimed is:

1. A method for improving a sexual sensory disorder of a subject comprising applying to the anogenitals of a subject an effective amount of a composition comprising a therapeutically effective amount of pirenzepine, or a pharmaceutically acceptable salt thereof.

2. The method of claim 1, wherein the method comprises improving sensitivity of the anogenitals of the subject.

3. The method of claim 1, wherein the method comprises increasing the sensitivity of the anogenitals of the subject.

4. The method of claim 1, wherein the method comprises reducing the sensitivity of the anogenitals of the subject.

5. The method of claim 1, wherein the method comprises reducing an anogenital pain.

6. The method of claim 1, wherein the method reverses, prevents or slows a sexual function symptom.

7. The method of claim 1, wherein the composition is applied to the skin of the anogenitals.

8. The method of claim 1, wherein the subject is a male.

9. The method of claim 1, wherein the composition is applied to the penis and/or anus.

10. The method of claim 1, wherein the subject is a female.

11. The method of claim 1, wherein the composition is applied to the external genitalia, cervix, vagina, vulva, labium, introitus, clitoris, urethral meatus, urethral fold, vulval vestibule, perineum, and/or anus.

12. The method of claim 1, wherein the composition comprises pirenzepine, or a pharmaceutically acceptable salt at a concentration of between about 1% and 15% by weight.

13. The method of claim 1, wherein the composition comprises pirenzepine, or a pharmaceutically acceptable salt at a concentration of between about 1% and 6% by weight.

14. The method of claim 1, wherein the composition is formulated for topical, transdermal or transmucosal administration.

15. The method of claim 1, wherein the composition is in a gel, cream, lotion, solution, suspension, emulsion, ointment, powder, crystalline form, spray, foam, salve, paste, plaster, paint, bioadhesive, suppository, tampon, body wipe, wound dressings, skin patches, genital pads, or transdermal patch.
